(19) United States
(12) Patent Application Publication
(10) Pub. No.: US 2009/0264345 A1
(43) Pub. Date: Oct. 22, 2009

(54) MACROCYCLIC PEPTIDES AND METHODS FOR MAKING AND USING THEM

(75) Inventors: Shelli R. McAlpine, San Diego, CA (US); Kathleen McGuire, Jamul, CA (US)

Correspondence Address: MORRISON & FOERSTER LLP, 12531 HIGH BLUFF DRIVE, SUITE 100, SAN DIEGO, CA, 92130-2040, US

(73) Assignee: San Diego State University Research Foundation, San Diego, CA (US)

(21) Appl. No.: 12/282,156

(22) PCT Filed: Mar. 19, 2007
(86) PCT No.: PCT/US07/64317

§ 371 Date: Mar. 24, 2009

Related U.S. Application Data
(60) Provisional application No. 60/783,298, filed on Mar. 17, 2006.
(60) Provisional application No. 60/797,111, filed on May 2, 2006.

(51) Int. Cl. A61K 38/12; C07K 7/64; A61P 35/00; A61P 17/06; A61P 19/02; A61P 27/06

(52) U.S. Cl. 514/11; 530/321

(57) ABSTRACT

The invention provides novel macrocyclic peptides and methods for their preparation. The invention also provides pharmaceutical compositions and methods to treat, prevent or ameliorate a cell proliferative disease or conditions. e.g., a cancer. in a subject in need thereof, including but not limited to a colon cancer, such as MSS or MSI colon cancer, and pancreatic cancer. This invention provides for the synthesis and development of novel anticancer agents that are based on the core structure Sansalvamide A (San A).

### CLAIM FOR PRIORITY

This application claims priority from U.S. Provisional Application Ser. No. 60/783,298, filed 17 Mar. 2006; U.S. Provisional Application Ser. No. 60/797,111, filed 2 May 2006; and U.S. Utility Application Ser. No. 11/436,378, filed 17 May 2006. The contents of each of these documents is expressly incorporated by reference herein in their entirety for all purposes.

### FIELD OF THE INVENTION

The present invention relates to the field of organic chemistry and medicine. The invention provides novel macrocyclic compounds based on the core structure of Sansalvamide A (San A), and methods for their preparation and use. In particular, compounds of the present invention comprise cyclic pentapeptides, their pharmaceutically acceptable salts and hydrate forms, and derivatives thereof, and pharmaceutical formulations comprising these compositions. Such compounds possess anticancer activity and activity comprising anti-cell-proliferative, anti-cell migration and/or apoptotic (promoting) activity, and are therefore useful in methods of treatment of a human or an animal body. The invention also relates to processes for the manufacture of said cyclic pentapeptides, to pharmaceutical compositions comprising them, and to their use in the manufacture of medicaments for use in the production of an anticancer effect in a warm-blooded animal such as man. The invention also provides methods of using said cyclic pentapeptides or pharmaceutical compositions to treat, prevent (prophylaxis of) or ameliorate cancers. including, but not limited to, colon cancer such as MSS and MSI colon cancers, pancreatic cancer, rectal cancer, breast cancer, prostate cancer, and melanoma.

### BACKGROUND

Sansalvamide A (San A) is a lipophilic depsipeptide marine natural product isolated from a marine fungus (Fusarium ssp.), which has been shown to exhibit cytotoxic activity in several cancer cell lines. (Fenical et al., Tetrahedron Lett. 1999, 40, 2913-16). In a mechanism of action study in the poxvirus molluscum contagiosum virus (MCV), San A was shown to be an inhibitor of a virus-encoded topoisomerase I. (Hwang et al., Molecular Pharmacology 1999, 55, 1049-1053). Unlike most Topo I inhibitors, San A does not stabilize Topo I-DNA covalent complexes. Rather, it inhibits the binding of the enzyme to DNA, most likely by interacting directly with Topo I. (Hwang, et al., Molecular Pharmacology 1999, 55, 1049-1053; Dias, et al., Top Curr Chem 2005, 253, (89-108). However, it is not known if San A inhibits mammalian topoisomerase, nor if its ability to inhibit cell growth is due to this mechanism. Without wishing to be bound by theory, it is possible that San A and its pentapeptide analogs demonstrate their anticancer effects by inhibition of topoisomerase activity.

In general, the macrocyclic peptides exhibit favorable biological, chemical, and physical properties. Cyclic peptides are more hydrophobic and are able to penetrate cell membranes faster than linear peptides, thus providing improved oral availability. Further, the cyclic nature of these compounds restricts bond rotation and provides a more rigid three-dimensional structure than linear peptides or other small molecule drugs. Finally, cyclic peptides are resistant to degradation by proteases, leading to longer half-lives in vivo. Such compounds are useful for developing therapeutic agents because of their diverse functionalization, defined three-dimensional conformations, and extended half-lives compared to linear peptides.

San A is composed of four hydrophobic amino acids and one hydrophobic hydroxy-acid. Compounds of the present invention are cyclic pentapeptide analogs of San A, where the hydroxy acid in position 4 is exchanged for an amino acid.

Colon carcinoma is one of the most common human cancers; pancreatic cancer is somewhat less common but more lethal than colon cancer. Both diseases have been considered for years as among the most drug resistant types of cancers. Pancreatic cancer is the fifth most deadly cancer in the U.S. Only 10% of patients are eligible for surgery, fewer than 20% of pancreatic cancers respond to the drug of choice (2,2-difluorodeoxycytidine; Gemzar), and the mortality rate is 95% in 5 years. Recently several new drugs, specifically oxaliplatin, bevucizumab, cetuximab and the tyrosine kinases inhibitors, have improved survival; however, current therapy is far from acceptable. In spite of significant research efforts, few truly novel classes of compounds have been identified that have activity against these 2 types of tumors. Thus, there is an urgent medical need to develop more effective drugs for the treatment of colon and pancreatic cancer.

Carcinogenesis in the colon/rectum is thought to occur through two different pathways. The existing model suggests that 80-85% of colon cancers involve chromosomal instability, where point mutations are found in loci within RAS, p53, and other checkpoint proteins. (Boland et al., Gastroenterology 2000, 118 S115-S128; Carethers et al., Gastroenterology 1999, 117, 123-131). The remaining 15-20% of colon cancers involve a loss in the DNA mismatch repair system, which leads to point mutations in repetitive sequences. These repetitive sequences are known as microsatellites, and occur in several important growth regulators. Mutations in these repetitive sequences lead to instability within microsatellites, which ultimately impacts the function of these growth regulator proteins. The two pathways are usually referred to as having microsatellite stability (MSS), or microsatellite instability (MSI) respectively. MSI colon cancers are resistant to current chemotherapeutic drugs and MSS colon cancers are treated with a relatively toxic drug.

Currently, only the MSS colon cancers are known to respond to chemotherapeutic drugs. The drug of choice for treatment, 5-fluorouracil (5-FU) [IC₅₀=5 μM], has significant side effects, making it desirable to develop a drug with improved efficacy. Because MSI colon cancers do not respond to 5-FU, or to current chemotherapeutic drugs, finding new structures that target both cancer pathways would be very valuable.

The mechanism of action of San A in the MSS and MSI cell lines is not completely understood. However, San A is known to inhibit Topoisomerase I activity, which is important for DNA replication, repair, and transcription. (Hwang et al., Molecular Pharmacology 1999, 55, 1049-1053).

Heat shock protein, Hsp90, functions as a molecular chaperone for intracellular signaling molecules. There are two isoforms of Hsp90, alpha and beta. Because it folds, assembles, and stabilizes proteins that regulate the growth of cancer cells, both Hsp90 isoforms are up-regulated in most cancers. There are 3 distinct regions of Hsp90: the N-terminal domain, the C-terminal domain, and the middle domain. Both isoforms exist as homodimers that are connected via the C-terminal region. The N-terminal domain contains the ATP binding site, which is the binding site for compounds targeting Hsp90 that are currently in clinical trials. Inhibitors of Hsp90 successfully stop cancer cell growth; thus they have outstanding potential as anticancer therapeutics.

A recent U.S. patent application by Silverman, US 2005/0159346, describes cyclic pentapeptides having antitumor activity. All of the compounds described by Silverman et al. contain only L-amino acids and have a highly conserved sequence of cyclo[-Phe-Leu-Val-Leu-Leu-] or cyclo[-pBrPhe-Leu-Val-Leu-Leu-], which are N-methylated on at most one amino acid position. Silverman and co-workers proposed that the N-methyl moieties were responsible for activity in cancer cell lines. See Liu et al., J. Med. Chem. 2005; 48:3630-38.

The synthesis and cytotoxicity of some novel cyclic pentapeptides comprising all D- or all L-amino acids have been reported. (Carroll et al., Org. Lett. 2005; 7:3481-3484).

### SUMMARY

The invention provides novel cyclic pentapeptides, and methods for their preparation and use as anti-cell proliferative and/or anticancer agents; thus, the invention also provides pharmaceutical preparations and formulations comprising compounds of this invention. In one aspect, compounds of the present invention comprise cyclic peptides related to San A, which have a cyclic peptide backbone comprising five amino acid residues. In one aspect, compounds of the invention represent a novel structural class that targets cancers, including pancreatic cancers, colon cancers such as MSS and MSI forms of colon cancer, and other cancers and cell proliferative conditions.

The invention provides pharmaceutical compositions and formulations comprising one or more compositions of this invention, e.g., the cyclic pentapeptides of this invention, and a pharmaceutically acceptable excipient. Such compositions are useful for the treatment of cell proliferative diseases and conditions, such as cancers, for example, pancreatic cancer and colon cancer, e.g., MSS colon cancer or MSI colon cancer, rectal cancer, breast cancer, prostate cancer, and/or melanoma.

In one aspect, the invention provides a cyclic pentapeptide of formula (I):

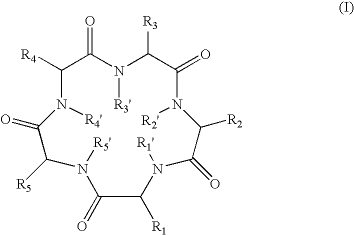

or a pharmaceutically acceptable salt or hydrate form thereof; and including any stereoisomers thereof;

wherein each of R_{1′}, R_{2′}, R_{3′}, R_{4′}, and R_{5′} independently represents H, or C1-C4 alkyl; and wherein R_{1′} may cyclize with R₁ to form a 5-10 membered azacyclic ring;

R₁ represents a C1-C4 alkyl, C5-C12 arylalkyl, C5-C12 heteroarylalkyl, or C1-C6 aminoalkyl group, each of which may be optionally substituted; or R₁ may cyclize with R_{1′} to form a 5-10 membered azacyclic ring; and

each of R₂, R₃, R₄, and R₅ independently represents H, or C1-C8 alkyl, C2-C8 alkenyl, C2-C8 alkynyl, C1-C8 cycloalkylalkyl, C1-C8 heterocyclylalkyl, C1-C6 aminoalkyl, C5-C 12 arylalkyl, or a heteroform of one of these, each of which may be optionally substituted;

with the proviso that the compound of formula (I) is not cyclo[-Phe-Leu-Val-Leu-Leu-] or cyclo[-pBrPhe-Leu-Val-Leu-Leu-], or a mono-N-methyl derivative thereof.

In another aspect, the invention provides a cyclic peptoid of formula (II):

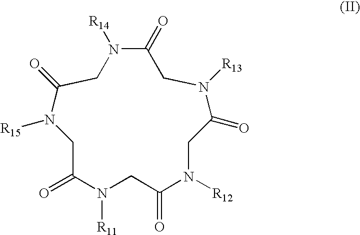

or a pharmaceutically acceptable salt or hydrate form thereof;

wherein each of R₁₁, R₁₂, R₁₃, R₁₄ and R₁₅ is independently selected from H, or C1-C8 alkyl, C2-C8 alkenyl, C2-C8 alkynyl, C1-C6 aminoalkyl, C5-C12 arylalkyl, or a heteroform of one of these, each of which may be optionally substituted.

In another aspect, the invention provides pharmaceutical compositions comprising one of more cyclic pentapeptides of formula (I) and (II), or a pharmaceutically acceptable salt or hydrate form thereof, and at least one pharmaceutically acceptable excipient.

In another aspect, the invention provides processes for the manufacture of cyclic pentapeptides of formula (I) and to pharmaceutical compositions comprising them, and to their use in the manufacture of medicaments for use in the production of an anticancer effect in a warm-blooded animal such as man.

In another aspect, the invention provides methods of treating, ameliorating or preventing (prophylaxis of) (including preventing a recurrence of) a cancer, such as colon cancers, e.g., MSS or MSI colon cancers, pancreatic cancer, rectal cancer, breast cancer, prostate cancer, and melanoma, by administering to a patient in need thereof a therapeutically effective amount of a pharmaceutical composition of formula (I) or formula (II).

In yet another aspect, the invention provides modified forms of compounds of formula (I) and (II), including peptides labeled with biotin or a fluorophoric groups, such as rhodamine, as well as peptides coupled to stabilizing or targeting agents, and to methods of using these compounds and formulations. In certain embodiments, biotinylated peptides of the present invention are useful for example in affinity assays. Fluorescently labeled peptides are useful, for example, to study the mechanism of action of compounds of the invention. In specific embodiments, biotin or rhodamine are linked to a lysine residue in the cyclic pentapeptides backbone via an alkylene or heteroalkylene linkage.

In still another aspect, the invention provides methods to synthesize compounds of formula (I) and formula (II), and/or their pharmaceutically acceptable salt or hydrate forms.

In a further aspect, the invention provides kits comprising compositions of the invention (e.g., the pharmaceutical compositions, formulations), including instruction means for practicing the methods of the invention.

The present invention provides novel cyclic pentapeptides comprising both D- and L-amino acids in their cyclic backbone. These cyclic pentapeptides generally have good aqueous solubility and enhanced stability over their linear counterparts. Additionally, the incorporation of unnatural amino acid residues into the cyclic peptide backbones, particularly the inclusion of D-amino acids, further enhances their stability against proteases.

In one aspect, the compounds of the invention possess a unique chemical structure and represent a novel class of anticancer and anti-cell growth/cell proliferative therapeutic agents. In one aspect, these compounds demonstrate cytotoxicity against a variety of cancer cell lines. In one aspect, compounds of the invention are cytotoxic against cancer cells, including colon cancer such as colon cancer MSS cells and cell lines, colon cancer MSI cells and cell lines (chemotherapeutically resistant strains), pancreatic cancer cells and cell lines, rectal cancer cells and cell lines and breast cancer cells and cell lines. Notably, compounds of the invention demonstrate cytotoxicity against chemotherapeutically resistant MSI colon cancer cells and pancreatic cancer cells, difficult-to-cure cancers for which no effective treatments are currently available. These cyclic pentapeptides demonstrate cytotoxicity comparable to 5-FU against MSS colon cancer. Further, the cyclic pentapeptides are also potent against MSI colon cancer.

Using affinity chromatography experiments, biotin-labeled cyclic pentapeptides of the present invention were surprisingly found to bind to Hsp90. In particular, compounds of the invention appear to bind to a unique region on the C-terminus of Hsp90. Without wishing to be bound by theory, compounds of the present invention may demonstrate their anticancer effects by interaction with Hsp90, a well-established oncogenic, representing an innovative approach towards treatment of these cancers.

In one aspect, the compounds of the present invention target drug-resistant cancers, e.g., colon and pancreatic cancer cells. In one aspect, incorporation of a single N-methyl and/or a single D-amino acid leads to significantly improved potency against a cancer, e.g., a colon and pancreatic cancer cell in vivo or a cell lines. In one aspect, compounds wherein a single L-amino acid is exchanged with a D-amino acid at amino acid residue 2 and/or 3 and/or 5 exhibited significantly enhanced potency against colon cancer cell lines relative to the corresponding peptides comprising all-L amino acids (including those that comprise N-methyl moieties) or all-D amino acids. San A derivatives comprising a single D-amino acid exhibit excellent “drug-like” potency as antitumor agents, and this structure-activity relationship (SAR) is general for the two types of colon cancers (MSS and MSI).

The invention provides a use of at least one compound of the invention, or a compound made by a method of the invention, for the preparation of a pharmaceutical or a veterinary composition.

The invention provides a use of at least one compound of the invention, or a compound made by a method of the invention, for the preparation of a pharmaceutical or a veterinary composition to treat, ameliorate or prevent a skin condition, psoriasis, a hormone-dependent tumor or a hormone-influenced non-malignant disorder, benign prostate hyperplasia (BPH), endometriosis; a disease or condition having an inflammatory component, an autoimmune disease, rheumatoid arthritis, an infectious disease, diabetic retinopathy, neovascular glaucoma, rheumatoid arthritis, psoriasis, a cancer, a lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma and/or any combination thereof.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The following drawings are illustrative of embodiments of the invention and are not meant to limit the scope of the invention as encompassed by the claims.

FIG. 1 shows representative examples of cyclic pentapeptides which comprise only L-amino acid residues in their cyclic peptide backbones.

FIG. 2 shows representative examples of cyclic pentapeptides which comprise only D-amino acid residues in their cyclic peptide backbones.

FIG. 3 shows representative examples of cyclic pentapeptides which comprise one D-amino acid residue and four other amino acid residues in their cyclic peptide backbones.

FIG. 4 shows representative examples of cyclic pentapeptides which comprise more than one D-amino acid residues and one or more other amino acid residues in their cyclic peptide backbones.

FIG. 5 shows representative examples of additional compounds of the invention, including cyclic pentapeptides which comprise all-L amino acids, as well as examples having one or more D-amino acid residues.

FIG. 6 shows the inhibitory activities of various cyclic pentapeptides against HT-29 (MSS colon), SW-480 (MSS colon), HCT-116 (MSI colon), and PL-45 (pancreatic) cancer cell lines.

FIG. 7 shows compounds with changes at position 1 run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 8 shows compounds with changes at position 2 run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 9 shows compounds with changes at position 3 run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 10 shows compounds with changes at position 4 run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 11 shows compounds with changes at position 5 run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 12 shows compounds comprising all L- or all D-amino acids combined with N-methylated amino acids run in three cell lines: HCT-116 and HCT-15 colon cancer and PL-45 pancreatic cancer cell lines. Data represents results from at least 3 separate experiments and each performed in quadruplicate. Margin of error is ±5%.

FIG. 13 shows IC₅₀s of compounds run in three cell lines: HCT-116 and HCT-15 (colon) and PL-45 (pancreatic). Data represents results from at least 3 separate experiments and each performed in quadruplicate. Margin of error is ±5%. 200 μM is the outside limit of detection.

FIG. 14 shows affinity assays using biotinylated compound (27). Lane 1=MW markers; lanes 2-5=0.1, 1, 10, 100 μM non-labeled (27) in colon cancer cell lysate HCT-116; lanes 6-9=0.1, 1, 10, 100 μM non-labeled (27) in pancreatic cancer cell lysate PL-45.

FIG. 15 shows results from the Annexin V assay. Panel 15(a) shows cells +1% DMSO only at 90 minutes. Panel 15(b) show cells +1% DMSO+50 μM compound (55) at 90 minutes.

FIG. 16 shows fluorophore-labeled compound (24) incubated with PL-45 cancer cells. Dapi was used to stain the nucleus and the picture using the Dapi wavelength filter was overlaid on top of the picture using the Rhodamine wavelength filter (16b). Panel 16(a) shows 24-fluorophore is visible in cells. Panel 16(b) shows a Dapi filtered view of cells overlaid on top of the Rhodamine filtered view.

Like reference symbols in the various drawings indicate like elements.

### DETAILED DESCRIPTION

The invention provides novel cyclic pentapeptides, and pharmaceutically acceptable formulations thereof, including for example their pharmaceutically acceptable salts or hydrate forms, and derivatives thereof. In one aspect, the compositions of the invention have anti-cancer activity, anti-cell-proliferative, anti-cell migration and/or apoptotic activity. Thus, compounds of the invention are useful in methods of treatment for a subject afflicted with any disease or condition comprising cell proliferation, e.g., a cancer or an infection that results in unwanted cell growth.

The invention also provides methods for the preparation of novel cyclic pentapeptides, and pharmaceutically acceptable formulations thereof, and to their use as pharmaceuticals, e.g., as anti-cell growth agents, as anticancer agents and the like. Use of the pharmaceuticals of the invention can be for ameliorating (treating) any disease or condition comprising cell proliferation, e.g., a cancer or an infection, or for ameliorating or preventing (prophylaxis of) (including preventing a recurrence of) their onset or recurrence, or for ameliorating or preventing side effects such as unwanted cell proliferation or hyperplasia.

The invention also provides processes for the manufacture of cyclic pentapeptides of this invention, to pharmaceutical compositions (e.g., formulations) comprising them and to their use in the manufacture of medicaments for use in the production of an anticancer, anti-cell-proliferation/migration and/or apoptotic effects in any individual, e.g., any warm-blooded animal such as man or animal, including veterinary uses.

The cyclic pentapeptides and pharmaceutical compositions of this invention can be useful to treat, prevent or ameliorate cancers, including, but not limited to, MSS and MSI colon cancers, pancreatic cancer, rectal cancer, breast cancer, prostate cancer, brain cancer, liver cancer, and/or melanoma or any other skin cancer, leukemias, and the like. The cyclic pentapeptides and pharmaceutical compositions of this invention can be useful to treat, prevent or ameliorate any cell proliferative condition, e.g., a skin condition such as psoriasis, or a hormone-dependent tumor or a hormone-influenced non-malignant disorder such as benign prostate hyperplasia (BPH) and endometriosis; or any disease or condition having an inflammatory component, e.g., an autoimmune disease such as rheumatoid arthritis, or an infectious disease; including treating, preventing or ameliorating any disease states associated with unwanted angiogenesis and/or cellular proliferation, such as diabetic retinopathy, neovascular glaucoma, rheumatoid arthritis, and psoriasis.

Cancers that can be treated, prevented or ameliorated by using compositions of this invention include lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and any combination thereof.

The present invention may be understood more readily by reference to the following detailed description of the preferred embodiments of the invention and the Examples included herein. However, before the present methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting. It is further to he understood that unless specifically defined herein, the terminology used herein is to be given its traditional meaning as known in the relevant art.

As used herein, the singular form “a”, “an”, and “the” include plural references unless indicated otherwise. For example, “a” peptide includes one of more peptides.

As used herein, the term “residue” refers to a particular amino acid that is incorporated into the pentapeptide backbone of the present invention.

For clarity, the amino acid residue bearing the substituent R₁ in formula (I) is sometimes referred to herein as “residue 1” or alternatively as “position 1”. Similarly, the amino acid residue bearing the substituent R₂ is sometimes referred to herein as “residue 2” or “position 2”, and so on around the pentapeptide ring, up to the amino acid bearing substituent R₅, which is sometimes referred to herein as “residue 5” or “position 5”.

In one aspect, the term “alkyl” includes straight-chain, branched-chain and cyclic monovalent hydrocarbyl radicals, and combinations of these, which contain only C and H when they are unsubstituted. Alkyl groups may be optionally unsaturated, such as in alkenyl or alkynyl groups. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it may be described as 1-10 C or as C1-C10 or as C1-10 or as C₁₋₁₀.

In one aspect, “alkenyl” and “alkynyl” groups are defined similarly to alkyl groups, and include straight-chain, branched-chain and cyclic monovalent hydrocarbyl radicals, and combinations of these, which contain only C and H when they are unsubstituted, However, alkenyl groups contain one or more carbon-carbon double bonds, and alkynyl groups contain one or more carbon-carbon triple bonds.

Typically, the alkyl, alkenyl and alkynyl substituents of the invention contain 1-8 C (alkyl) or 2-8 C (alkenyl or alkynyl). Preferably they contain 1-4 C (alkyl) or 2-4 C (alkenyl or alkynyl).

Alkyl, alkenyl and alkynyl groups are often substituted to the extent that such substitution makes sense chemically. Preferred substituents include, but are not limited to, halo, ═O, —CN, —OR′, ′SR′, —S(O)R′, —SO₂R′, —COOR′, —C(O)NR′₂, —NR′₂ and —NHC(═NH)NH₂, where each R′ independently represents H, C1-C4 alkyl or C5-C12 arylalkyl, or a heteroform of one of these.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain one or more heteroatoms selected from O, S and N and combinations thereof, within the backbone residue. When heteroatoms (typically N, O and S) are allowed to replace carbon atoms of an alkyl, alkenyl or alkynyl group, as in heteroalkyl groups, the numbers describing the group, though still written as e.g. C1-C6, represent the sum of the number of carbon atoms in the group plus the number of such heteroatoms that are included as replacements for carbon atoms in the ring or chain being described. Such heteroalkyl groups may be optionally substituted with the same substituents as alkyl groups.

Where such groups contain N, the nitrogen atom may be present as NH or it may be substituted if the heteroalkyl or similar group is described as optionally substituted. Where such groups contain S, the sulfur atom may optionally be oxidized to SO or SO₂ unless otherwise indicated. For reasons of chemical stability, it is also understood that, unless otherwise specified, such groups do not include more than two contiguous heteroatoms as part of the heteroalkyl chain, although an oxo group may be present on N or S as in a nitro or sulfonyl group. Thus —C(O)NH₂ can be a C2 heteroalkyl group substituted with ═O; and —SO₁NH— can be a C2 heteroalkylene, where S replaces one carbon, N replaces one carbon, and S is substituted with two ═O groups.

While “alkyl” in one aspect includes cycloalkyl and cycloalkylalkyl groups, the term “cycloalkyl” may be used herein to specifically describe a carbocyclic non-aromatic group that is connected via a ring carbon atom, and “cycloalkylalkyl” may be used to describe a carbocyclic non-aromatic group that is connected to the base molecule through an alkyl linker. For example, cyclohexylalanine (Cha) comprises a cycloalkylalkyl substituent. Similarly, “heterocyclyl” may be used to describe a non-aromatic cyclic group that contains at least one heteroatom as a ring member and that is connected to the molecule via a ring atom of the cyclic group, which may be C or N; and “heterocyclylalkyl” may be used to describe such a group that is connected to another molecule through an alkyl linker. The sizes and substituents that are suitable for the cycloalkyl, cycloalkylalkyl, heterocyclyl, and heterocyclylalkyl groups are the same as those described above for alkyl groups. Where an alkyl group is substituted with an aryl or heteroaryl group, it is referred to as an arylalkyl or heteroarylalkyl substituent.

In one aspect, an “aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, pyridazinyl, triazinyl, thienyl, furanyl, pyrrolyl, pyrazolyl, thiazolyl, isothiazolyl, oxazolyl, isoxazolyl, imidazolyl, triazolyl, thiadiazolyl, oxadiazolyl, and tetrazolyl rings, and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolinyl, quinolinyl, benzothiazolyl, benzofuranyl, benzothienyl, benzisoxazolyl, pyrazolopyridyl, quinazolinyl, quinoxalinyl, cinnolinyl, and the like.

Any monocyclic or fused ring bicyclic system which has the characteristics of aromaticity in terms of electron distribution throughout the ring system is included in this definition. It also includes bicyclic groups where at least one ring has the characteristics of aromaticity, even though it may be fused to a nonaromatic ring. Typically, the ring systems contain 5-12 ring member atoms. Preferably the monocyclic heteroaryl groups contain 5-6 ring members, and the bicyclic heteroaryls contain 8-10 ring members.

Aryl and heteroaryl moieties may be substituted with a variety of substituents which are known in the art. Preferred substituents include, but are not limited to, halo, C1-C8 alkyl, —NO₂, —CN, —OR′, —SR′, —COOR′, —C(O)NR′₂, and —NR′₂, where each R′ independently represents H, C1-C4 alkyl or C5-C12 arylalkyl, or a heteroform of one of these.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moieties. “Heteroarylalkyl” refers to a moiety comprising an aryl group that is attached through a linking group, and differs from “arylalkyl” in that at least one ring atom of the aryl moiety or one atom in the linking group is a heteroatom selected from N, O and S.

An aryl or heteroaryl ring in an arylalkyl or heteroarylalkyl group may be optionally substituted on the aromatic portion with the same substituents described above for aryl groups. In preferred embodiments, an arylalkyl group includes a phenyl ring and a heteroarylalkyl group includes a C5-C6 monocyclic or C8-C10 fused bicyctic heteroaromatic ring, each of which may be optionally substituted with the groups defined above for aryl groups and a C1-C4 alkylene that is unsubstituted or is substituted with one or two C1-C4 alkyl groups, where the alkyl groups can optionally cyclize to form a ring, and wherein the alkyl or heteroalkyl groups may be optionally fluorinated. In certain embodiments, the arylalkyl or heteroarylalkyl ring comprises a phenol or an indole ring. Preferred substituents on phenyl include OH, C1-C4 alkoxy, and halo.

“Arylalkyl” and “heteroarylalkyl” groups are described by the total number of carbon atoms in the ring and alkylene or similar linker. Thus a benzyl group is a C7-arylalkyl group, and phenethyl is a C8-arylalkyl group.

“Alkylene” in one aspect refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. However, for clarity, a three-atom linker that is an alkylene group, for example, refers to a divalent group in which the available valences for attachment to other groups are separated by three atoms such as —(CH₂)₃—, i.e., the specified length represents the number of atoms linking the attachment points rather than the total number of atoms in the hydrocarbyl group: —C(Me)₂— would thus be a one-atom linker, since the available valences are separated by only one atom. Where an alkylene group is substituted, the substituents include those typically present on alkyl groups as described herein, thus —C(═O)— is an example of a one-carbon substituted alkylene. Where it is described as unsaturated, the alkylene may contain one or more double or triple bonds.

“Heteroalkylene” in one aspect is defined similarly to the corresponding alkylene groups, but the ‘hetero’ terms refer to groups that contain one or more heteroatoms selected from O, S and N and combinations thereof, within the backbone residue; thus at least one carbon atom of a corresponding alkylene group is replaced by one of the specified heteroatoms to form a heteroalkylene group. Thus, —C(═O)NH— is an example of a two-carbon substituted heteroalkylene, where N replaces one carbon, and C is substituted with a ═O group.

In one aspect, an “aminoalkyl” group refers to a C1-C6 alkyl group that is substituted with at least one amine group having the formula —NR2, where each R is independently H, C1-C8 alkyl, C5-C12 aryl and C5-C12 arylalkyl, or a heteroform of one of these. Such aminoalkyl groups may be optionally substituted on the alkyl portion with one or more other groups suitable as substituents for an alkyl group. In some embodiments, the aminoalkyl substituent is a 1-aminoalkyl group such as a 1-aminomethyl, 1-aminoethyl, 1-aminopropyl or 1-aminobutyl group. In certain embodiments, the aminoalkyl group may comprise a protected amine. One of skill in the art would appreciate that appropriate amine protecting groups may vary depending on the functionality present in the particular monomer. Suitably protected amines may include, for example, carbamates (e.g. tert-butoxycarbonyl, benzyloxycarbonyl, fluorenylmethyloxycarbonyl, allyloxycarbonyl or (trialkylsilyl)ethoxycarbonyl), carboxamides (e.g. formyl, acyl or trifluoroacetyl), sulfonamides, phthalimides, Schiff base derivatives, and the like. In certain embodiments, an aminoalkyl group may be coupled through an alkylene or heteroalkylene linker to a group such as biotin, or a fluorophore-containing group, such as rhodamine, and such compounds may be useful for screening or mechanistic studies.

“Heteroform” in one aspect refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Optionally substituted” in one aspect indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Halo”, in one aspect includes fluoro, chloro, bromo and iodo. Fluoro and chloro are often preferred.

“Amino” in one aspect refers to NR′₂ wherein each R′ is independently H, or is an alkyl, alkenyl, alkynyl, acyl, aryl, or arylalkyl group or a heteroform of one of these groups, as defined above, each of which may be optionally substituted with the substituents described herein as suitable for the corresponding type of group. In certain embodiments, the two R′ groups on one nitrogen atom may be linked together to form an azacyclic ring.

In one aspect, an ‘azacyclic’ group refers to a heterocyclic group containing at least one nitrogen atom as a ring atom, wherein the group is attached to the base molecule through a nitrogen atom of the azacyclic group. Typically azacyclic groups are 3-8 membered monocyclic rings or 8-12 membered bicyclic fused ring systems, and may be saturated, unsaturated or aromatic and may contain a total of 1-3 heteroatoms independently selected from N, O and S as ring members. In certain embodiments, an azacyclic ring may comprise a nitrogen-containing ring fused to a phenyl ring. For example, the unnatural amino acid “Tic” comprises a tetrahydroisoquinoline ring, which represents a 10-membered fused bicyclic azacyclic group.

In one aspect, a “therapeutically effective amount” means that amount of a drug or pharmaceutical agent that will elicit desired therapeutic effect, biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In one aspect, “subject” refers to a human or other warm-blooded animal subject.

In one aspect, “peptide” and “polypeptide” are used interchangeably and refer to a compound made up of a chain of amino acid residues linked by peptide bonds. Unless otherwise indicated, the sequence for peptides is given in the order from the amino terminus to the carboxyl terminus. In certain embodiments, one or more amino acids in the peptide are D-amino acids.

In one aspect, the cyclic pentapeptides of the invention have the structures and/or sequences described herein with at least one conservative amino acid substitution, where such compounds retain their activity, e.g., retain their anticancer activity and activity comprising anti-cell-proliferative, anti-cell migration and/or apoptotic (promoting) activity. In one aspect, the “conservative amino acid substitutions” are substitutions which do not result in a significant change in the activity or tertiary structure of a selected polypeptide or protein. In one aspect, the substitutions typically involve replacing a selected amino acid residue with a different residue having similar physico-chemical properties. Groupings of amino acids by physico-chemical properties are known to those of skill in the art. In one aspect, the “conservative amino acid substitutions” comprises exchange of residues between families of amino acid residues having similar side chains have been defined in the art, and include basic side chains (e.g., lysine, arginine, histidine), acidic side chains (e.g., aspartic acid, glutamic acid), neutral polar side chains (e.g., asparagine, cysteine, glutamine, serine, threonine, tyrosine), neutral nonpolar side chains (e.g., alanine, glycine, isoleucine, leucine, methionine, phenylalanine, proline, tryptophan, valine), beta-branched side chains (e.g., isoleucine, threonine, valine) and aromatic side chains (e.g., tyrosine, phenylalanine, tryptophan, histidine). Thus, the substitution of leucine by isoleucine would be a conservative substitution, because both contain neutral nonpolar side chains, whereas the substitution of leucine by lysine would be non-conservative, because the neutral nonpolar side chain of Leu is replaced by the basic sidechain of Lys.

Generally, the nomenclature used herein and the laboratory procedures in analytical chemistry, organic chemistry, material sciences, and nanotechnology described herein are those well known and commonly employed in the art. Standard techniques, or modifications thereof, are used for chemical syntheses and chemical analyses of the present invention. (See generally, March, “ADVANCED ORGANIC CHEMISTRY: REACTIONS, MECHANISMS, AND STRUCTURE”, 3rd ed. (1985) John Wiley & Sons, New York, N.Y.)

In one aspect, unless otherwise explicitly stated, references herein to cyclic pentapeptides are also meant to include their stereoisomers, pharmaceutically acceptable salts and hydrate or solvate forms, as well as pharmaceutical compositions and formulations thereof. Additionally, therapeutically active metabolites, where the metabolites themselves fall within the scope of the claimed invention, are also compounds of the current invention. Prodrugs, which are compounds that are converted to the claimed compounds as they are being administered to a patient or after they have been administered to a patient, are also compounds of this invention.

In one aspect, the cyclic pentapeptides of the present invention have a cyclic peptide backbone which comprises five amino acid residues. The compounds of the invention can comprise natural and/or unnatural amino acids. Suitable unnatural amino acids can include, but are not limited to, D-amino acids and N-alkylated amino acids, especially N-methylated amino acids. D-amino acids may be denoted herein as, for example, _{D}Xaa, D-Xaa or (D)-Xaa, whereas N-alkylated amino acids may be referred to herein as NR″Xaa, where R″ corresponds to the N-alkyl substituent and Xaa corresponds to the particular amino acid residue. For example, N-methyl valine may sometimes be referred to herein as NMeVal, and (D)-valine may sometimes be referred to herein as _{D}Val.

In one aspect, the term “amino acid” is used in the conventional sense to refer to an organic chemical compound comprising at least one amino group (i.e., —NH₂ or —NR_{N}H) and at least one carboxylic acid group (i.e., —COOH). In some cases, an amino group may be a substituted amino group (i.e., —NR_{N}H, where R_{N} is a nitrogen substituent), for example, as in the case of proline. For convenience, amino acids are often denoted herein as AA, or as H—AA—OH, where the initial —H is part of an amino group, and the final —OH is part of a carboxylic acid group. Amino acids may often be conveniently further classified according to their structure, for example, as alpha-amino acids, beta-amino acids, and the like.

In one aspect, the term “alpha amino acid” is used in the conventional sense to refer to amino acids in which at least one carboxylic acid group (i.e., —COOH) and at least one amino group (i.e., —NH₂ or —NR_{N}H) are directly attached to a single carbon atom (designated the alpha carbon) and may be conveniently denoted HNR_{N}—CR_{A}R_{B}—COOH, wherein R_{N}, R_{A} and R_{B} are substituents. Two or more of the substituents R_{N}, R_{A} and R_{B} may together form a single multivalent substituent, thus a cyclic alpha amino acid. For example, in the cyclic alpha-amino acid proline, R_{N} and R_{A} together form the single divalent substituent —CH₂CH₂CH₂—, and R_{B} is —H.

If the substituents R_{A} and R_{B} are different, the alpha carbon will be chiral (i.e., R or S), and the alpha-amino acid will be optically active. For example, glycine, for which R_{A} and R_{B} are both —H, is not optically active, whereas alanine, for which R_{A} is —CH₃ and R_{B} is —H, is optically active and may be in D- or L-forms, denoted D-alanine or L-alanine, respectively. The alpha carbon of D-alanine is in the R configuration whereas the alpha carbon of L-alanine is in the S configuration.

Of the wide variety of alpha-amino acids known, only about twenty are naturally occurring. Naturally occurring alpha-amino acids are often denoted HNR_{N}—CHR—COOH (since R_{B} is —H) where R_{N} denotes a nitrogen substituent and R denotes an amino acid substituent (often referred to as an amino acid sidechain). The nitrogen substituent R_{N} is —H for all naturally occurring alpha amino acids, with the exception of proline (where R_{N} and R together form the divalent substituent —CH₂CH₂CH₂—). Except for glycine, all of these twenty naturally occurring alpha-amino acids are optically active and are in the L-form. Examples of amino acid substituents include those substituents found in the twenty naturally occurring alpha amino acids, such as, for example, —H (glycine, G, Gly), —CH₃ (alanine, A, Ala), —CH₂OH (serine, S, Ser), —CH(CH₃)OH (threonine, T, Thr), CH₂SH (cysteine, C, Cys), and —CH₂C₆ H₅ (phenylalanine, F, Phe). Other examples of amino acid substituents include those which are structurally similar to those substituents found in the naturally occurring amino acids, such as, for example, —CH₂CH₂OH (homoserine) and —CH₂CH₂SH (homocysteine).

For convenience, the naturally occurring amino acids are often represented by a three letter code or a one-letter code. The three-letter and one-letter codes for the twenty naturally occurring acids are well established in the art, and the standard conventions are used herein. The following conventional three-letter amino acid abbreviations are used herein: Ala=alanine; Arg=arginine; Asn=asparagine; Asp=aspartic acid; Cys=cysteine; Gln=glutamine; Glu=glutamic acid; Gly=glycine; His=histidine; Ile=isoleucine; Leu=leucine; Lys=lysine; Met=methionine; Nle=norleucine; Orn=ornithine; Phe=phenylalanine; Phg=phenylglycine; Pro=proline; Sar=sarcosine; Ser=serine; Thr=threonine;Trp=tryptophan; Tyr=tyrosine; and Val=valine.

In addition to an alpha carboxylic acid group (i.e., —COOH) and an alpha amino group (i.e., —NH₂ or —NR_{N}H), many amino acids have additional functional groups. Lysine, for which the amino acid substituent, R, is —(CH₂)₄NH₂, has an additional amino group (i.e., —NH₂). Aspartic acid and glutamic acid, for which the amino acid substituents, R, are —CH₂COOH and —(CH₂)₂COOH, respectively, each have an additional carboxylic acid group (i.e., —COOH). Serine, for which the amino acid substituent, R, is —CH₂OH, has an additional primary hydroxyl group (i.e., —OH). Threonine, for which the amino acid substituent, R, is CH(CH₃)OH, has an additional secondary hydroxyl group (i.e., —OH). Cysteine, for which the amino acid substituent, R, is —CH₂SH, has an additional thiol group (i.e., —SH). Other amino acids have other additional functional groups, including, for example, thioether groups (e.g., in methionine), phenol groups (e.g., in tyrosine), amide groups (e.g., in glutamine), and heterocyclic groups (e.g., in histidine).

In addition to the twenty naturally occurring amino acids, several other classes of alpha amino acids are also known. Examples of these other classes include D-amino acids, Nαalkyl amino acids, alpha-alkyl amino acids, cyclic amino acids, chimeric amino acids, and miscellaneous amino acids. These non-natural amino acids have been widely used to modify bioactive polypeptides to enhance resistance to proteolytic degradation and/or to impart conformational constraints to improve biological activity (Hruby et al., Biochem. J. (I 990) 268:249-262; Hruby and Bonner, Methods in Molecular Biology (1994) 35:201-240). The most common Nα-alkyl amino acids are the Nα-methyl amino acids, such as, Nα-methyl glycine (i.e., NMeGly, sarcosine, Sar), Nα-methyl alanine (i.e., NMeAla), and Nα-methyl lysine (i.e., NMeLys). Also included herein are other Nα-methyl amino acids including Nα-methyl valine (i.e., NMeVal), Nα-methyl leucine (i.e., NMeLeu), and Nα-methyl phenylalanine (i.e., NMePhe). Examples of alpha-alkyl amino acids include alpha-aminoisobutyric acid (i.e., Aib), diethylglycine (i.e., Deg), diphenylglycine (i.e., Dpg), alpha-methyl proline (i.e., ((αMe)Pro), and alpha-methyl valine (i.e., (αMe)Val) (Balaram, Pure & Appl. Chem. (1992) 64:1061-1066; Toniolo et al., Biopolymers (1993) 33:1061-1072; Hinds et al., J. Med. Chem. (1991) 34:1777-1789). Examples of cyclic amino acids include 1-amino-1-cyclopropane carboxylic acid, 1-amino-1-cyclopentane carboxylic acid (i.e., cyclic leucine), aminoindane carboxylic acid (i.e., Ind), tetrahydroisoquinolinecarboxylic acid (i.e., Tic) and tetrahydrocarbolinecarboxylic acid (i.e., Tca) (Toniolo, C., Int. J. Peptide Protein Res. (1990) 35:287-300; Burgess, K., Ho, K. K., and Pal, B. J. Am. Chem. Soc. (1995) 117:3808-3819). Also included are alkenyl and alkynyl containing amino acids such as propargylglycine, dehydroalanine, and the like. Examples of chimeric amino acids include penicillamine (i.e., Pen), combination of cysteine with valine, and 4-mercaptoproline (i.e., Mpt), combination of proline and homocysteine. Example of miscellaneous alpha-amino acids include ornithine (i.e., Orn), 2-naphthylalanine (i.e., 2-Nal), phenylglycine (i.e., Phg), t-butylglycine (i.e., tBug), alpha-ethylglycine (i.e., (αEt)Gly), alpha-n-propylglycine (i.e., (αPr)Gly), alpha-n-butylglycine (i.e., nBug), O-benzylserine (i.e., (OBzl)Ser), p-bromophenylalanine (i.e., pBrPhe), cyclohexylalanine (i.e., Cha), and alpha-amino-2-thiophenepropionic acid (i.e., Thi).

In addition to alpha-amino acids, others such as beta amino acids, can also be used in the present invention. Examples of these other amino acids include 2-aminobenzoic acid (i.e., Abz), .beta-aminopropanoic acid (i.e., .beta-Apr), .gamma-aminobutyric acid (i.e., gamma-Abu), and 6-aminohexanoic acid (i.e., epsilon-Ahx).

The cyclic pentapeptide of the present invention may be synthesized by synthesizing a linear peptide with the same peptide sequence and then cyclizing the linear peptide. In the synthesis and manipulation of amino acid-containing species (e.g., polypeptides), it is often necessary to “protect” certain functional groups (such as alpha-amino groups, alpha-carboxylic acid groups, and side-chain functional groups) of amino acids. A wide variety of protecting groups and strategies are known in the art. For example, an alpha-amino group (i.e., —NH2) may be protected with a 9 fluorenylmethyloxycarbonyl group (i.e., Fmoc; as —NHFmoc), a tertbutoxycarbonyl group (i.e., —C(═O)OC(CH3)3, Boc; as —NHBoc), or a benzyloxycarbonyl group (i.e., —C(═O)OCH2C6H5, CBZ; as —NHCBZ). The guanidino group of arginine (i.e., —NHC(═NH)NH2) may be protected with a 2,2,5,7,8-pentamethylchroman-6-sulfonyl group (i.e., Pmc; as —NHC(═NH) —NH—Pmc), a 4-methoxy-2,3,6-trimethylbenzenesulfonyl group (i.e., Mtr; as —NHC(═NH) —NH—Mtr), or a mesitylene-2-sulfonyl group (i.e., Mts; as —NHC(═NH)—NH—Mts). The carboxamide groups of asparagine and glutamine (i.e., —CONH2) may be protected with a trityl group (i.e., —C(C6H5)3, Tr; as —CONHTr). The side chain carboxylic acid groups of aspartic and glutamic acid may be protected with a t-butyl group (i.e., —C(CH3)3, tBu; as —COOtBu) or a cyclohexyl group (i.e., —C6H11, cHx; as —COOcHx). Additionally, carboxylic acid groups, such as terminal carboxylic acid groups, may be protected with a methyl group (i.e., —CH3, as —COOCH3). an ethyl group (i.e., —CH2CH3, as COOCH2CH3), or a benzyl group (i.e., —CH2C6H5, as —COOCH2C6H5). The thiol group of cysteine (i.e., —SH) may be protected with a t-butylthio group (i.e., —SC(CH3)3, tBuS; as —SStBu) or a trityl group (i.e., —C(C6H5)3, Tr; as —STr). The imidazole group of histidine may be protected with a trityl group (i.e., —C(C6H5)3. Tr; as —STr). The epsilon-amino group of lysine (i.e., —NH2) may be protected with a tert-butoxycarbonyl group (i.e., —C(═O)OC(CH3)3, Boc as —NHBoc), a benzyloxycarbonyl group (i.e., —C(═O)OCH2C6H5, CBZ; as —NHCBZ), or a 2-chlorobenzyloxycarbonyl group.(i.e., —C(═O)OCH2C6H4Cl, 2—Cl-CBZ; as —NH—2Cl—CBZ). The hydroxyl groups of homoserine, serine and threonine (i.e., —OH) may be protected with a t-butyl group (i.e., —C(CH3)3, tBu; as —OtBu), a trityl group (i.e., —C(C6H5)3, Tr; as —OTr), or a t-butyldimethylsilyl group (i.e., —Si(CH3)2(C(CH3)3), TBDMS; as —OTBDMS). The indole nitrogen of tryptophan may be protected with a trityl group (i.e., —C(C6H5)3, Tr). The hydroxyl group of tyrosine (i.e., —H) may be protected with a trityl group (i.e., —C(C6H5)3, Tr; as —OTr).

The peptide linkage (i.e., —C(═O) —NRₙ—) of a polypeptide may conveniently be considered to be the chemical linkage formed by reacting a carboxylic acid group (i.e., —COOH) of one amino acid with an amino group (i.e., —NR_{R}H) of another amino acid. In this way, a polypeptide (e.g., a “2-mer”) of the two amino acids serine and cysteine (wherein the carboxylic acid group of serine and the amino group of cysteine have formed a peptide linkage) may conveniently be represented as H—Ser—Cys—OH or H—S—C—OH, or, more simply, as Ser—Cys, S—C, or SC. The amino acid moieties of a polypeptide are often referred to as amino acid residues.

In one aspect, the invention provides a compound of formula (I):

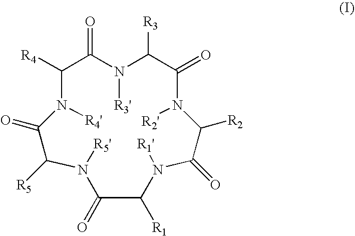

or a pharmaceutically acceptable salt or hydrate form thereof.

For compounds of formula (I), each of R_{1′}, R_{2′}, R_{3′}, R_{4′}, and R_{5′} independently represents H, or C1-C4 alkyl or C1-C4 heteroalkyl. In many embodiments, each of R_{1′}, R_{2′}, R_{3′}, R_{4′}, and R_{5′} independently represents H or methyl. In preferred embodiments, any one of R_{1′}, R_{2′}, R_{3′}, R_{4′}, and R_{5′} is methyl, and the other four of R_{1′}, R_{2′}, R_{3′}, R_{4′}, and R_{5′} are H. In other preferred embodiments, each of R_{1′}, R_{2′}, R_{3′}, R_{4′}, and R_{5′} is H. In certain embodiments, R_{1′} may cyclize with R₁ to form a 5-10 membered azacyclic ring. In some embodiments, R_{1′} may cyclize with R₁ to form a tetrahydroisoquinoline ring.

For compounds of formula (I), R₁ represents a C5-C12 arylalkyl, C5-C12 heteroarylalkyl, or C1-C6 aminoalkyl group, each of which may be optionally substituted. In certain embodiments, R₁ represents an optionally substituted C5-C12 arylalkyl group. In preferred embodiments, R₁ represents CH₂ArX, where Ar represents a phenyl ring and X is selected from H, halo, OH and C1-C4 alkoxy. In some embodiments, R₁ may cyclize with R_{1′} to form a 5-10 membered azacylic ring.

For compounds of formula (I), each of R₂, R₃, R_{4,} and R₅ independently represents H, or C1-C8 alkyl, C2-C8 alkenyl, C2-C8 alkynyl, C1-C8 cycloalkylalkyl, C1-C6 aminoalkyl, C5-C12 arylalkyl, or a heteroform of one of these, each of which may be optionally substituted. In certain embodiments, each of R₂, R₃, R₄, and R₅ independently represents H, methyl, ethyl, n-propyl, isopropyl, n-butyl, sec-butyl, isobutyl, tert-butyl, CH₂-cyclohexyl, CH₂OH, CH₂OBzl, or CH₂ArX, where Ar represents a phenyl ring and X is selected from H, halo, OH, and C1-C4 alkoxy.

In certain embodiments, the carbon atom bearing R₁ has the (R)-configuration and the carbon atoms bearing R₂, R₃, R₄, and R₅ have the (S)-configuration. In some such embodiments, R₁ is CH₂ArX, where Ar represents a phenyl ring and X is selected from H, OH, OMe, Br, Of Cl.

In some embodiments, R₂ is sometimes a C1-C4 alkyl or C5-C12 arylalkyl group. In certain embodiments, the carbon atom bearing R₂ has the (R)-configuration and the carbon atoms bearing R₁, R₃, R₄, and R₅ have the (S)-configuration. In some such embodiments. R₂ is benzyl or isobutyl, and R_{2′} is H or Me. In preferred embodiments, the carbon atom bearing R₂ has the (R)-configuration and comprises a phenyl ring, optionally substituted with OH, Br or Cl.

In some embodiments, R₃ is a C1-C4 alkyl group. In certain embodiments, R₃ is isopropyl. In preferred embodiments, the carbon atom bearing R₃ has the (R)-configuration. In further embodiments, the carbon atom bearing R₃ has the (R)-configuration and the carbon atoms bearing R₁, R₂, R₄, and R₅ have the (S)-configuration. In some such embodiments, R₃ is isopropyl or isobutyl, and R_{3′} is H or Me.

In further embodiments, R₄ is a C1-C4 alkyl group. In preferred embodiments, R₄ comprises a hydrophobic group.

In some embodiments, R₅ is a C1-C4 alkyl group. In preferred embodiments, R₅ is an isobutyl group. In certain embodiments, the carbon atom bearing R₅ has the (R)-configuration. In further embodiments, the carbon atom bearing R₅ has the (R)-configuration and the carbon atoms bearing R₁, R₂, R₃, and R₄ have the (S)-configuration. In some such embodiments, R5 is isopropyl or isobutyl, and R₅ is H or Me.

In certain embodiments, two or more of the carbon atoms bearing R₁, R₂, R₃, R₄ and R₅ have the (R)-configuration. In some embodiments, each of the carbon atoms bearing R₁, R₂, R₃, R₄ and R₅ has the (R)-configuration.

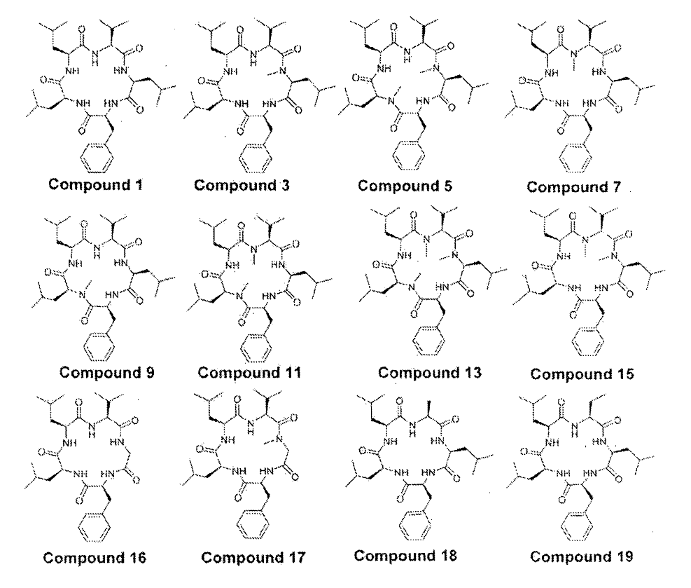

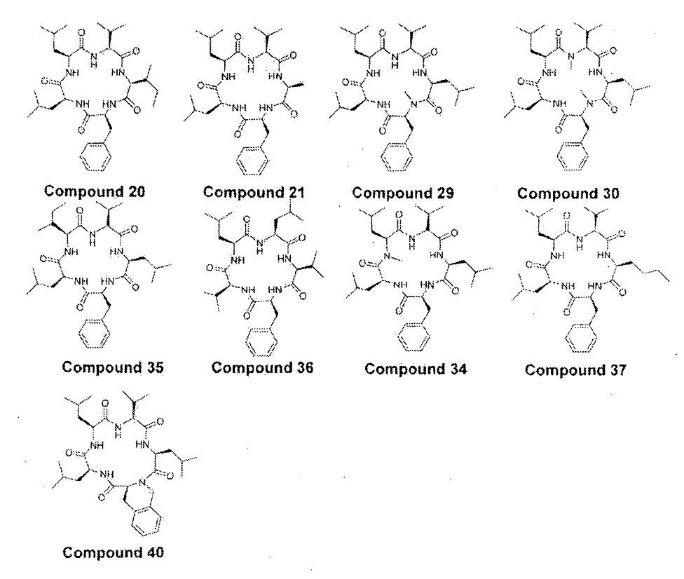

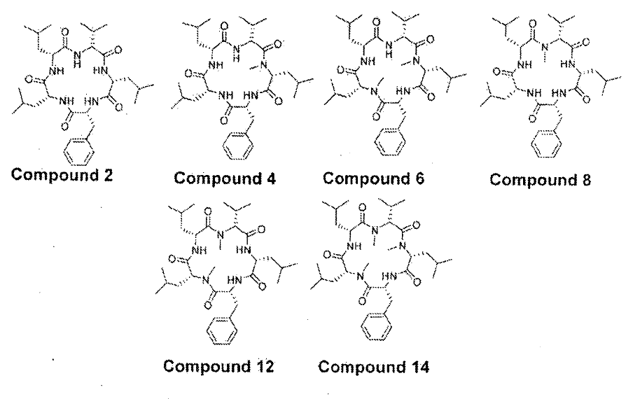

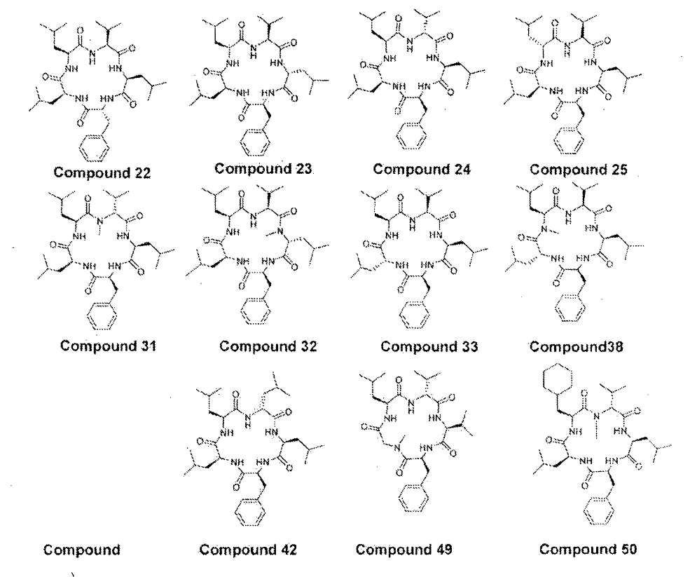

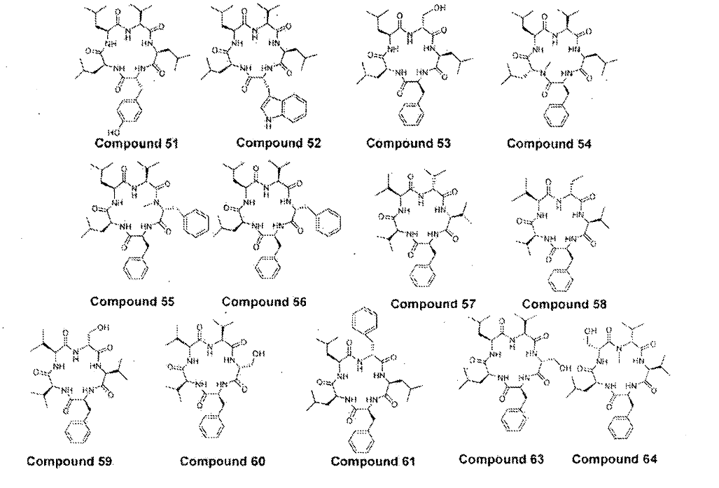

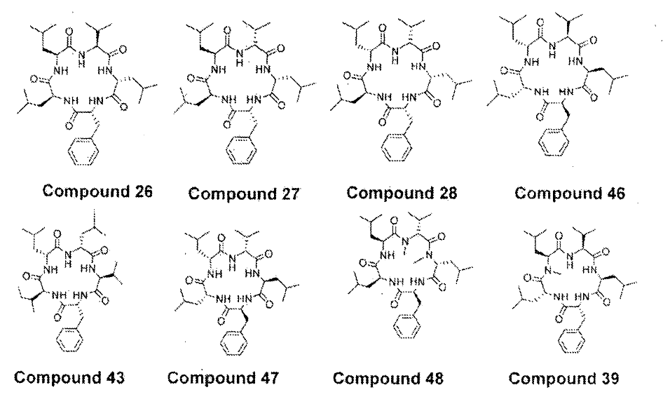

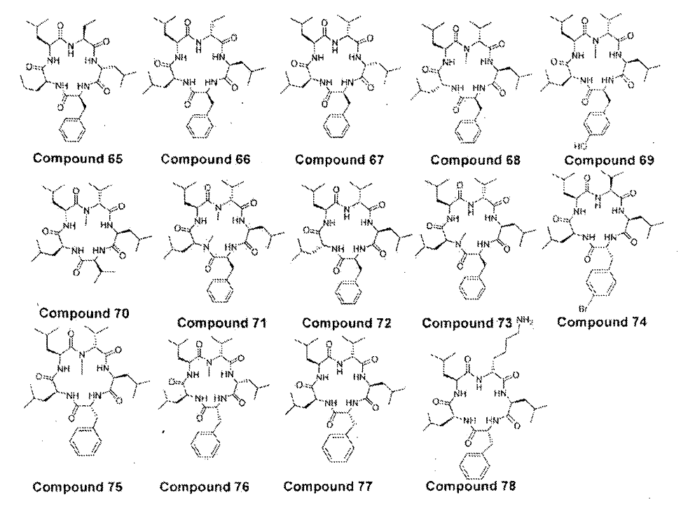

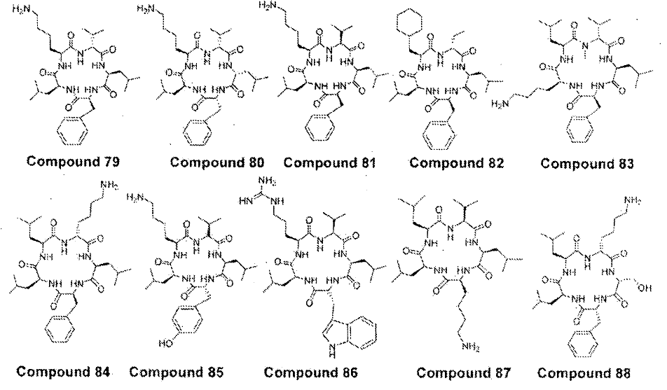

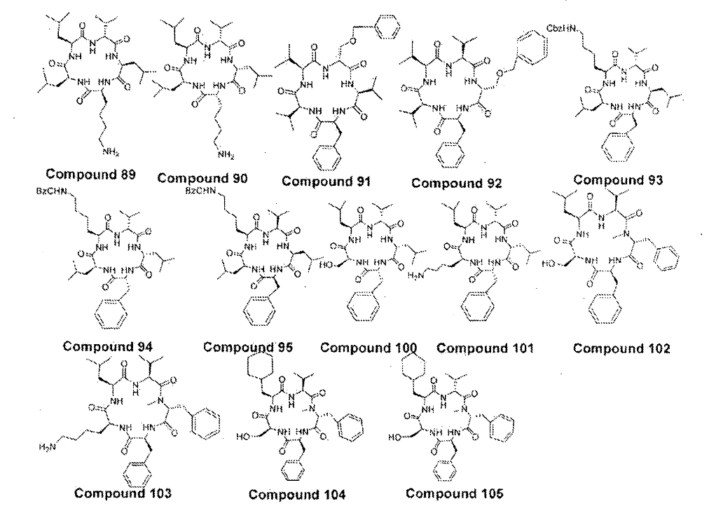

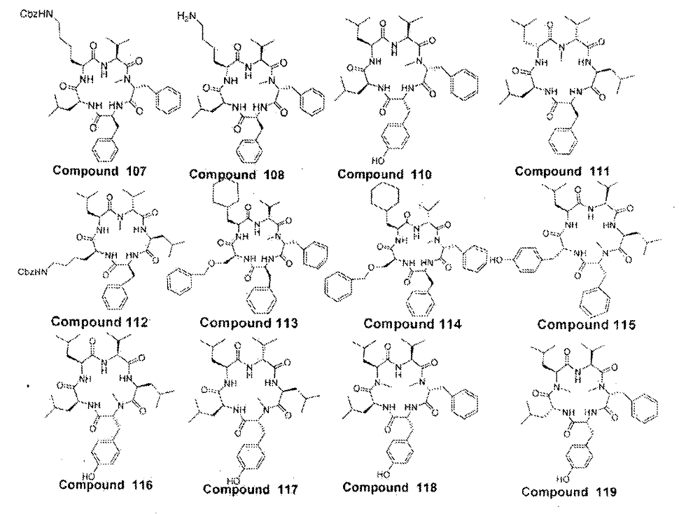

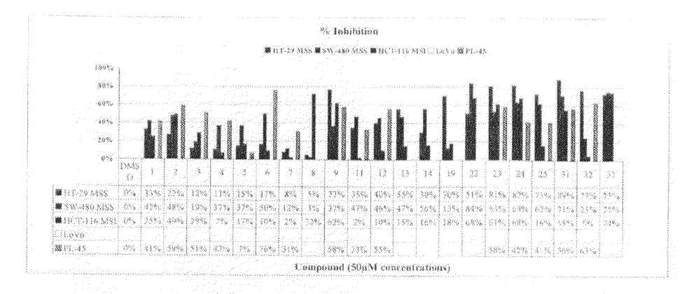

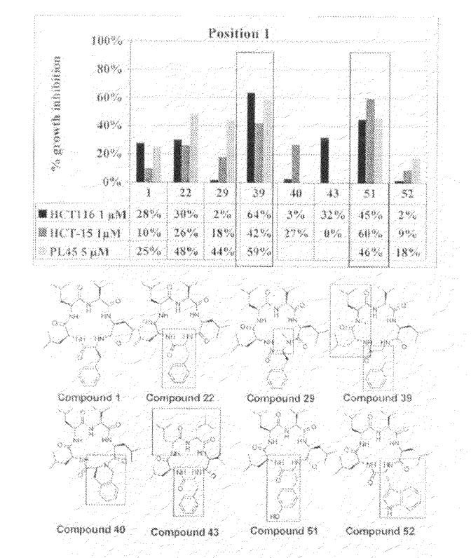

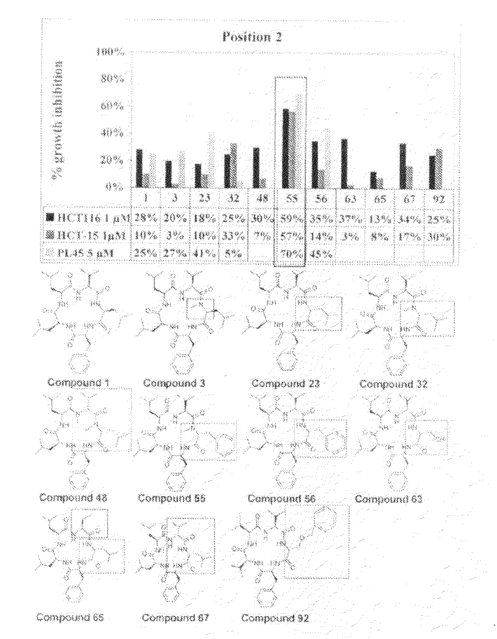

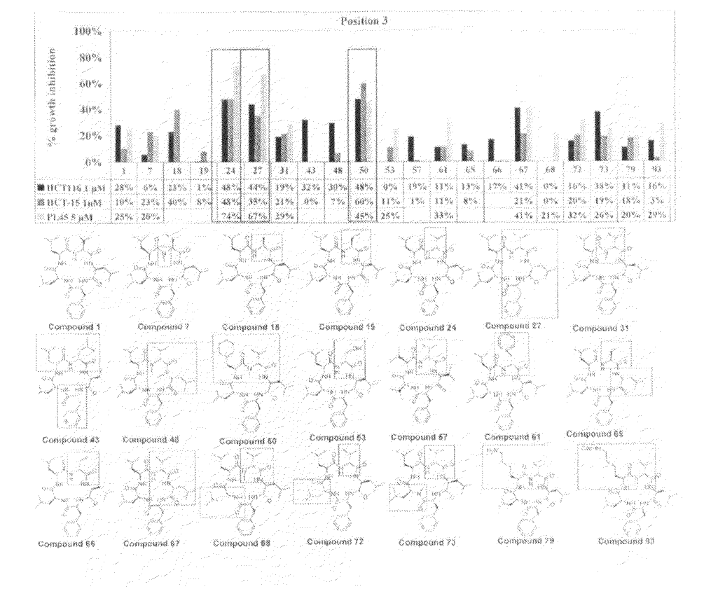

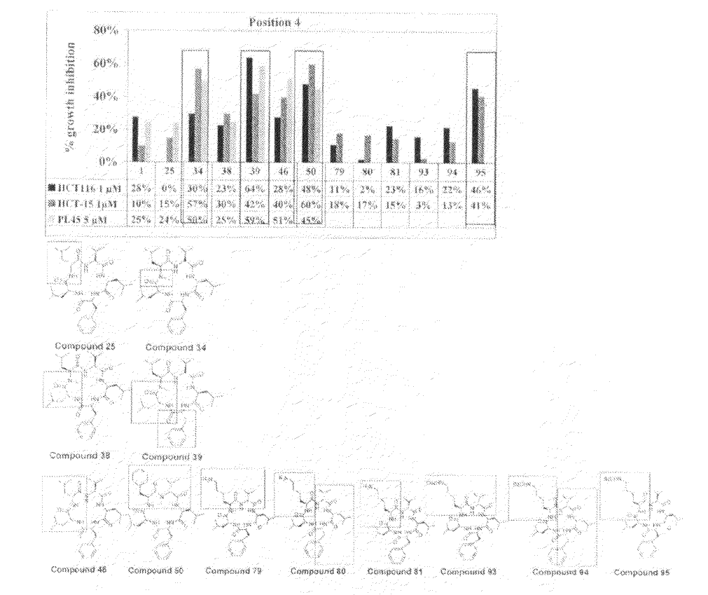

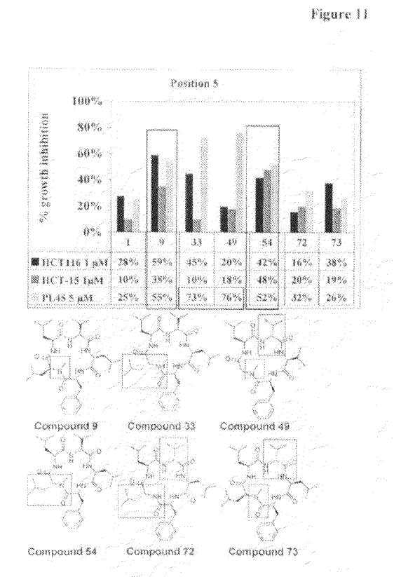

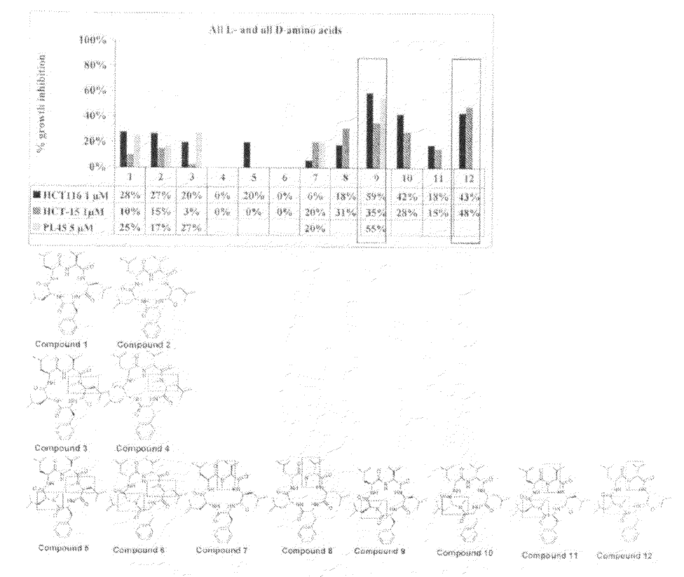

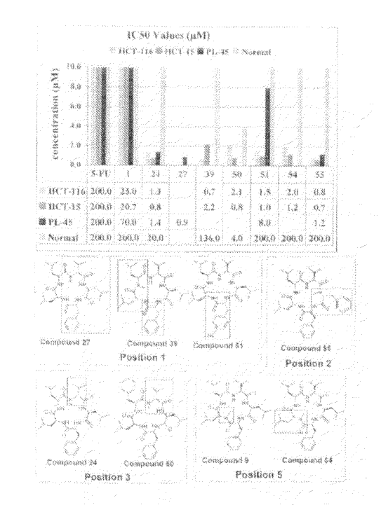

In one embodiment, the cyclic pentapeptide of formula (I) comprises only L-amino acid residues in its cyclic peptide backbone. Nonlimiting examples of cyclic L-pentapeptides are shown in FIG. 1.

In another embodiment, the cyclic pentapeptide of formula (I) comprises only D-amino acid residues in its cyclic peptide backbone. Nonlimiting examples of cyclic D-pentapeptides are shown in FIG. 2.

In an alternative embodiment, the cyclic pentapeptide of formula (I) comprises both D-and L-amino acid residues in the cyclic peptide backbone. The cyclic pentapeptide may comprise one, two, three, or four D-amino acid residues in the cyclic peptide backbone.

Nonlimiting examples of cyclic pentapeptides of formula (I) having a single D-amino acid residue are shown in FIG. 3. Non-limiting examples having two or more D-amino acids are shown in FIG. 4.

Additional non-limiting examples of cyclic pentapeptides comprising all L-amino acids, or comprising one or more D-amino acid residues in the cyclic backbone are shown in FIG. 5.

In certain preferred embodiments, the compounds of formula (I) comprise a single D-amino acid and four L-amino acids in the cyclic peptide backbone. In some such embodiments, the (D)-amino acid comprises residue 1, residue 2 or residue 3. In some embodiments comprising a single D-amino acid, R₁ comprises an optionally substituted C5-C12 arylalkyl group; in specific embodiments, residue 1 is (D)-tyrosine or (D)-phenylalanine. In other embodiments, R₂ comprises an optionally substituted C5-C12 arylalkyl group; in specific embodiments residue 2 is (D)-N-methyl-phenylanine. In further embodiments, R₃ comprises an optionally substituted C1-C4 alkyl group; in specific embodiments residue 3 is (D)-valine or (D)-N-methyl-valine. In further embodiments, R₅ comprises an optionally substituted C1-C4 alkyl group; in specific embodiments, residue 5 is N-methyl-leucine or (D)-N-methyl-leucine.

In certain embodiments, the compound of formula (I) comprises two or more D-amino acids and the remainder L-amino acids. In specific embodiments, residues 1 and 5 comprise D-amino acids. In other embodiments, residues 4 and 5 comprise D-amino acids.

In another aspect, the invention provides a cyclic pentapeptoid of formula (II):

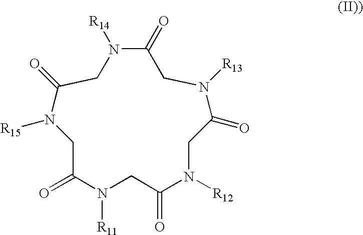

or a pharmaceutically acceptable salt or hydrate form thereof.

For compounds of formula (II), each of R₁₁, R₁₂, R₁₃, R₁₄ and R₁₅ is independently selected from H, or C1-C8 alkyl, C2-C8 alkenyl, C2-C8 alkynyl, C1-C8 cycloalkylalkyl, C1-C6 aminoalkyl, C5-C12 arylalkyl, or a heteroform of one of these, each of which may be optionally substituted.

In certain embodiments, each of R₁₁ and R₁₅ comprises a C5-C12 arylalkyl or C1-C8 cycloalkylalkyl group; each of R₁₂ and R₁₃ comprises a C1-C8 alkyl or C5-C12 arylalkyl group; and R₁₄ comprises a C1-C8 alkyl, C1-C8 heterocyclylalkyl, or C5-C12 arylalkyl group.

In another aspect, the invention provides pharmaceutical compositions comprising one of more cyclic pentapeptides of formula (I) or cyclic pentapeptoids of formula (II), or their pharmaceutically acceptable salt or hydrate forms, and at least one pharmaceutically acceptable excipient.

In another aspect, the invention provides methods of treating, preventing or ameliorating a cell proliferative disease or condition, e.g., a cancer, comprising providing a pharmaceutical composition of the invention; and administering a therapeutically effective amount of a pharmaceutical composition of formula (I) or a pharmaceutical composition of formula (II) to a patient in need thereof, thereby treating the desired condition, as described herein.

In a specific embodiment, this invention encompasses methods of treating, preventing, or ameliorating cancer using compounds or compositions of the invention, or pharmaceutically acceptable salts, solvates, hydrates, stereoisomers, or prodrugs thereof. In particular, the invention provides pharmaceutical compositions and methods of using such compositions to treat cancers, including but not limited to, colon cancers such as MSS colon cancer or MSI colon cancer, pancreatic cancer, rectal cancer, breast cancer, prostate cancer, and melanoma; or any cell proliferative condition, such as benign prostate hyperplasia (BPH) and endometriosis; or any disease or condition having an inflammatory component, e.g., an autoimmune disease such as rheumatoid arthritis, or an infectious disease; including treating, preventing or ameliorating any disease states associated with unwanted angiogenesis and/or cellular proliferation, such as diabetic retinopathy, neovascular glaucoma, rheumatoid arthritis, and psoriasis.

The invention also provides methods to use the compounds and pharmaceutical compositions of the invention in the manufacture of medicaments for use in the production of an anti-cell proliferative effect, e.g., an anti-anticancer effect, in a warm-blooded animal such as man or animal (a veterinary indication). Uses can be to treat or prevent, or prevent recurrence, or ameliorate symptoms, of any cell proliferative condition, such as a cancer, a benign prostate hyperplasia (BPH), endometriosis; an inflammatory or autoimmune disease such as rheumatoid arthritis, an infectious disease, unwanted angiogenesis and/or cellular proliferation, diabetic retinopathy, neovascular glaucoma, rheumatoid arthritis and/or psoriasis.

In yet another aspect, the invention provides modified forms of compounds of formula (I) and (II), including peptides and peptoids labeled with biotin or a fluorophoric groups, such as rhodamine, as well as peptides coupled to stabilizing or targeting agents, and to methods of making and using these compounds and formulations. In particular biotinylated peptides of the present invention are useful for affinity assays. Fluorescently labeled peptides are useful for studying the intracellular localization of compounds of the invention.

In still another aspect, the invention provides methods to synthesize compounds of formula (I) and formula (II), and/or their pharmaceutically acceptable salt or hydrate forms by macrocyclization of a linear pentapeptide or linear pentapeptoid precursor.

Furthermore, the invention provides kits comprising at least one composition of formula (I) or formula (II) (e.g., the pharmaceutical compositions or dietary supplements of the invention), including instruction means for practicing the methods of the invention (e.g., directions as to indications, dosages, routes and methods of administration).

The cyclic pentapeptides described herein may have asymmetric centers. It is understood, that whether a chiral center in an isomer is “R” or “S” depends on the chemical nature of the substituents of the chiral center. All configurations of compounds of the invention are considered part of the invention. Compounds of the present disclosure containing an asymmetrically substituted atom may be isolated in optically active or racemic forms. It is well known in the art how to prepare optically active forms, such as by resolution of racemic forms or by synthesis from optically active starting materials. Mixtures of isomers of the compounds of the examples or chiral precursors thereof can be separated into individual isomers according to methods which are known per se, e.g. fractional crystallization, adsorption chromatography or other suitable separation processes. Resulting racemates can be separated into antipodes in the usual manner after introduction of suitable salt-forming groupings, e.g. by forming a mixture of diastereoisomeric salts with optically active salt-forming agents, separating the mixture into diastereomeric salts and converting the separated salts into the free compounds. The enantiomeric forms may also be separated by fractionation through chiral high pressure liquid chromatography columns.

Many geometric isomers of olefins and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

In certain embodiments of the present invention, the cyclic pentapeptide is provided as a pharmaceutically acceptable salt for enhancing pharmacological properties. The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts”, in one aspect, refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic, manganous, potassium, sodium; zinc, and the like. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared fiom pharmaceutically acceptable non-toxic acids, including inorganic and organic acids. Such acids include acetic, 2-acetoxybenzoic, benzenesulfonic, benzoic, camphorsulfonic, citric, ethane disulfonic, ethanesulfonic, fumaric, gluconic, glutamic, hydrobromic, hydrochloric, isethionic, lactic, maleic, malic, mandelic, methanesulfonic, mucic, nitric, oxalic, pamoic, pantothenic, phosphoric, succinic, sulfamic, sulfuric, tartaric, p-toluenesulfonic acid, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418.

The invention provides parenteral formulations comprising a pharmaceutical composition of the invention. The invention provides enteral formulations comprising a pharmaceutical composition of the invention.

The invention provides methods for treating cancer comprising providing a pharmaceutical composition of the invention; and administering a therapeutically effective amount of the pharmaceutical composition to a subject in need thereof, thereby treating the desired condition, as described herein.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of Formula (I) or (II) for the treatment of, for example colon or pancreatic cancer, will generally be in the range of 0.1 to 100 mg/kg body weight of recipient (mammal) per day and more usually in the range of 1 to 10 mg/kg body weight per day. Thus. for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of Formula (I) or (II) per se. It is envisaged that similar dosages would be appropriate for treatment of the other conditions referred to herein.

In a specific embodiment, this invention encompasses methods of treating, preventing, and ameliorating, cancer using compounds of the invention, or pharmaceutically acceptable salts, solvates, hydrates, stereoisomers, or prodrugs thereof.

Cancers may be solid or blood-borne. Examples of cancer include, but are not limited to, cancers of the skin, such as melanoma; lymph node; breast; cervix; utenis; gastrointestinal tract; lung; ovary; prostate; colon; rectum; mouth; brain; head and neck; throat; testes; kidney; pancreas; bone; spleen; liver; bladder; larynx; nasal passages; AIDS-related cancers; endometrial tumors; sarcomas, e.g. soft tissue and bone sarcomas; and the hematological malignancies such as, e.g., leukemias. The compounds of the invention can be used for treating, preventing or ameliorating either primary or metastatic tumors. In particular embodiments, compounds of the invention are used to treat MSS and MSI colon cancers, pancreatic cancer, rectal cancer, breast cancer, prostate cancer, and melanoma.

In addition, compounds of the invention are also useful in the treatment of other cell proliferative disorders such as psoriasis, vascular smooth cell proliferation associated with atherosclerosis, post-surgical stenosis and restenosis, and in the treatment of Alzheimer's disease.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier. The pharmaceutical compositions used in the methods of the invention may be formulated in any way, and administered by any means known in the art.

Dosage forms

The pharmaceutical compositions of the present invention can be formulated in any way and can be administered in a variety of unit dosage forms depending upon the condition or disease and the degree of illness, the general medical condition of each patient, the resulting preferred method of administration and the like. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

Generally, the compounds may be used in an amount of from about 0.01 mg to about 2000 mg per day, and can be adjusted in a conventional fashion (e.g., the same amount administered each day of the treatment, prevention or management period), in cycles (e.g., one week on, one week off), or in an amount that increases or decreases over the course of treatment, prevention, or management. In other embodiments, the dose can be from about 0.1 mg to about 1000 mg, from about 0.1 mg to about 500 mg, from about 0.1 mg to about 100 mg, from about 0.1 mg to about 50 mg, from about 0.1 mg to 10 mg, from about 1 mg to about 1000 mg, from about 1 mg to about 500 mg, from about 1 mg to about 100 mg, from about 1 mg to about 50 mg, from about 1 mg to about 10 mg, from about 10 mg to about 1000 mg, from about 10 mg to about 500 mg, from about 10 mg to 100 mg, from about 10 mg to 50 mg, from about 50 mg to about 500 mg, from about 50 mg to 200 mg, or from about 100 mg to 300 mg per day.

If desired, the effective daily dose of the active compound may be administered as two, three, four, five, six or more sub-doses administered separately at appropriate intervals throughout the day, optionally, in unit dosage forms. In one embodiment, the compound is administered as one dose per day. In further embodiments, the compound is administered continuously, as through intravenous or other routes. In other embodiments, the compound is administered less frequently than daily, such as weekly or less.

While it is possible for a compound of the present invention to be administered alone, it is preferable to administer the compound as a pharmaceutical formulation (composition). The compound of the invention can be administered as such or in admixtures with pharmaceutically acceptable excipients, including for example diluents and/or carriers.

The subject receiving this treatment is any animal in need, including primates, in particular humans, and other mammals.

Formulations and Possible Routes of Administration

Details on techniques for formulation and administration are well described in the scientific and patent literature, see, e.g., the latest edition of Remington's Pharmaceutical Sciences, Maack Publishing Co, Easton Pa. (“Remington's”) (e.g., Remington, The Science and Practice of Pharmacy, 21st Edition, by University of the Sciences in Philadelphia, Editor).

Pharmaceutical formulations or dietary supplements may be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture, as further described herein.

In one aspect, invention provides a pharmaceutical composition or dietary supplements comprising compositions of the invention formulated as a tablet, gel, geltab, pill, implant, liquid, spray, powder, food, feed pellet, as an injectable formulation or as an encapsulated formulation, lotion, patch or inhalant. In one aspect, compositions of the invention can be chemically modified to produce a protected form that possesses better specific activity, prolonged half-life, and/or reduced immunogenicity in vivo, e.g., the composition can be chemically modified formulated or modified by glycosylation, pegylation (modified with polyethylene glycol (PEG), activated PEG, or equivalent), encapsulation with liposomes or equivalent, encapsulated in nanostructures (e.g., nanotubules, nano- or microcapsules), or combinations thereof, or equivalents thereof, e.g., as described by Wang (2005) Mol Genet Metab. 86(1-2): 134-140. Epub 2005 Jul. 11. In one aspect, the polypeptide is chemically conjugated with activated PEG, or, 2,4-bis(O-methoxypolyethyleneglycol)-6-chloro-s-triazine, e.g., as described by Ikeda (2005) Amino Acids 29(3):283-287. Epub 2005 Jun. 28.

If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The invention also provides biocompatible matrices such as sol-gels encapsulating a composition of the invention for use as pharmaceutical composition, e.g., including silica-based (e.g., oxysilane) sol-gel matrices. The invention also provides nano- or microcapsules comprising a composition of the invention for use as pharmaceutical composition or dietary supplements.

The pharmaceutical compositions and dietary supplements used in the methods of the invention can be administered by any means known in the art, e.g., parentelally, topically, orally, or by local administration, such as by aerosol or transdermally.

Pharmaceutical formulations and dietary supplements can be prepared according to any method known to the art for the manufacture of pharmaceuticals and dietary supplements. Such drugs and dietary supplements can contain sweetening agents, flavoring agents, coloring agents and preserving agents. A formulation (which includes “dietary supplements”) can be admixtured with nontoxic pharmaceutically or orally acceptable excipients which are suitable for manufacture.

Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations and dietary supplements for oral administration can be formulated using pharmaceutically acceptable carriers well known in the art in appropriate and suitable dosages. Such carriers enable the pharmaceuticals and dietary supplements to be formulated in unit dosage forms as tablets, pills, powder, dragees, capsules, liquids, lozenges, gels, syrups, slurries, suspensions, etc., suitable for ingestion by the patient. Pharmaceutical preparations and dietary supplements for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, lice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

Aqueous suspensions of the invention can an active agent comprising a composition of the invention in admixture with excipients suitable for the manufacture of aqueous suspensions. Such excipients include a suspending agent, such as sodium carboxymethyl-cellulose, methylcellulose, hydroxypropylmethylcellulose, sodium alginate, polyvinyl-pyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity. Dispersions can also be prepared in glycerol, liquid polyethylene glycols and mixtures thereof in oils. Under ordinary conditions of storage and use, these preparations contain a preservative to prevent the growth of microorganisms.

Oil-based pharmaceuticals are particularly useful for administration of hydrophobic formulations or active agents of the invention (a composition of the invention). Oil-based suspensions can be formulated by suspending an active agent (e.g., a composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations and dietary supplements can be preserved by the addition of an antioxidant such as ascorbic acid. As an example of an injectable oil vehicle, see Minto (1997) J. Pharmacol. Exp. Ther. 281:93-102.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Compounds of the invention may be administered intravenously. Depending on the patient, the type and stage of cancer, the type of chemotherapy, and the dosage selected, intravenous chemotherapy may be given on either an inpatient or outpatient basis. For continuous, frequent or prolonged intravenous chemotherapy administration, various systems may be surgically inserted into the vasculature to maintain access. Commonly used systems include the Hickman line, the Port-a-Cath or the PICC line. These systems result in a lower infection risk, reduce the incidence of phlebitis or extravasation, and abolish the need for repeated insertion of peripheral cannulae.

In the methods of the invention, the pharmaceutical compounds and dietary supplements can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

The pharmaceutical compounds, formulations and dietary supplements of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include the equivalent of lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. No. 20040028670.

The compositions (e.g., formulations, including dietary supplements) of the invention can be delivered by the use of liposomes. By using liposomes, particularly where the liposome surface carries ligands specific for target cells, or are otherwise preferentially directed to a specific organ, one can focus the delivery of the active agent into target cells in vivo. See. e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

### Combination Therapy

As described above, the compounds of the present invention may administered either as single agents or, alternatively, in combination with known anticancer treatments such as radiation therapy or a chemotherapy regimen in combination with cytostatic or cytotoxic agents. For example, the above compounds can be administered in combination with one or more chemotherapeutic agents, including but not limited to, taxanes (e.g., paclitaxel, Taxol®, docataxel), topoisomerase I inhibitors (e.g., camptothecin, topotecan, irinotecan), CPT-11, anthracycline glycosides (e.g., daunorubicin, doxorubicin or epirubicin), topoisomerase II inhibitors (e.g., etoposide, teniposide), vinca alkaloids (e.g., navelbine, vinblastine, vinblastine, vindesine, vinorelbine), nucleoside agents (e.g., gemcitabine, fluroruracil (5-FU), capecitabine, cytarabine, floxuridine, fludaribine), platinum-containing alkylating agents (e.g., carboplatin, cisplatin, oxiplatin), alkylating agents (e.g., nitrogen mustards, nitrosoureas), kinase inhibitors (e.g., dasatinib, erlotinib, gefitinib, imantinib, and the like), monoclonal antibodies (e.g., alemtuzumab, bevacizumab, cetuximab, gemtuzumab, rituxumab, and the like), optionally within liposomal formulations thereof.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). In one aspect additional therapeutic agents which are normally administered to treat or prevent a particular disease, or condition, are known as “appropriate for the disease, or condition, being treated”.

A combination treatment of the present invention as defined herein may be achieved by way of the simultaneous, sequential or separate administration of the individual components of said treatment.

Such other drugs may be administered by a route and in an amount commonly used therefor. When a compound of the invention is used contemporaneously with one or more other drugs, a pharmaceutical composition in unit dosage form containing such other drugs and the cyclic pentapeptide is preferred. However, the combination therapy also includes therapies in which the cyclic pentapeptide and one or more other drugs are administered on different schedules. When oral formulations are used, the drugs may be combined into a single combination tablet or other oral dosage form, or the drugs may be packaged together as separate tablets or other oral dosage forms. Accordingly, the pharmaceutical compositions of the present invention include those that contain one or more other active ingredients, in addition to a cyclic pentapeptide.

It is also contemplated that when used in combination with one or more other active ingredients, the compound of the present invention and the other active ingredients may be used in lower doses than when each is used singly. For example, the use of such combination therapies could provide additive or synergistic anticancer effects.

Combination of two or more drugs in therapy may result in one of three outcomes: (1) additive, i.e., the effect of the combination is be equal to the sum of the effects of each drug when administered alone; (2) synergistic, i.e., the effect of the combination is greater than the sum of the effects of each drug when administered alone; or (3) antagonistic, i.e., the effect of the combination is less than the sum of the effects of each drug when administered alone.

The effects of combinations of drugs are enhanced when the ratio in which they are supplied provides a synergistic effect. Synergistic combinations of agents have also been shown to reduce toxicity due to lower dose requirements, to increase cancer cure rates (Barierre, et al., Pharmacotherapy (1992) 12:397-402, Schimpff, et al., Support Care Cancer (1993) 1:5-8), and to reduce the spread of multi-resistant strains of microorganisms (Schlaes, et al., Clin. Infect. Dis. (1993) 17:S527-S536). By choosing agents with different mechanisms of action, multiple sites in biochemical pathways can be attacked thus resulting in synergy (Shah, et al., Clin. Cancer Res. (2001) 7:2168-2181). Combinations such as L-canavanine and 5-fluorouracil have been reported to exhibit greater antineoplastic activity in rat colon tumor models than the combined effects of either drug alone (Swaffar, et al., Anti-Cancer Drugs (1995) 6:586-593). Cisplatin and etoposide display synergy in combating the growth of a human small-cell lung-cancer cell line, SBC-3 (Kanzawa, et al., Int. J. Cancer (1997) 71(3):311-319).

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

### Synthesis of Cyclic Pentapeptides of Formula (I)

Compounds of formula (I) were synthesized via a convergent solution phase approach, as shown in Scheme 1.

This approach provides a reliable, high-yielding route for preparation of the cyclic pentapeptides of the present invention. The approach involves two fragments, a tripeptide fragment comprising amino acid residues 1, 2 and 3, and a dipeptide fragment comprising amino acid residues 4 and 5. The route is amenable to inserting desired amino acids systematically within the cyclic pentapeptide backbone. The route was also designed to facilitate large-scale synthesis for extensive biological studies. Cyctization to form large macrocycles is usually very challenging, and typically the yields are low. For the present invention, high-yielding cyclization conditions were developed which provided the final macrocycles in good yields.

The approach described in Scheme 1 is suitable for the preparation of cyclic pentapeptides containing diverse functionality, including for example aliphatic, aromatic and polai sidechains, as well as the incorporation of both natural and unnatural amino acids, including for example, D-amino acids and N-alkylated amino acids, at various positions around the pentapeptide backbone.

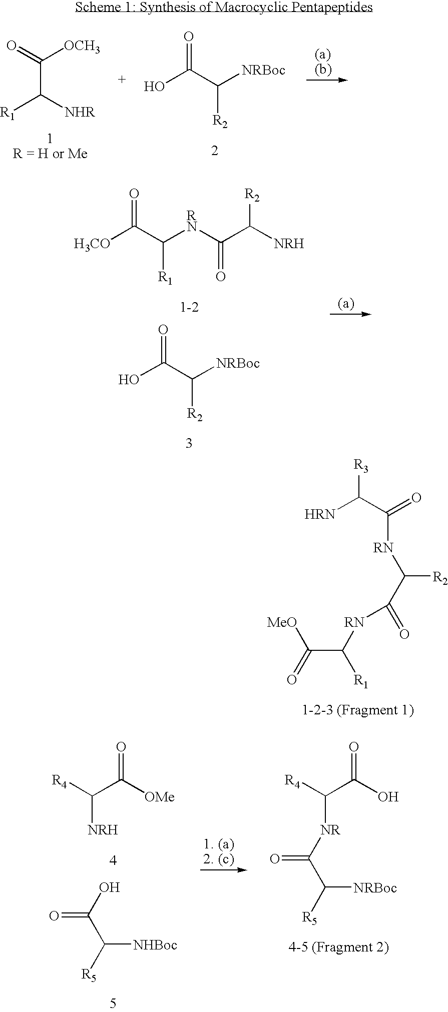

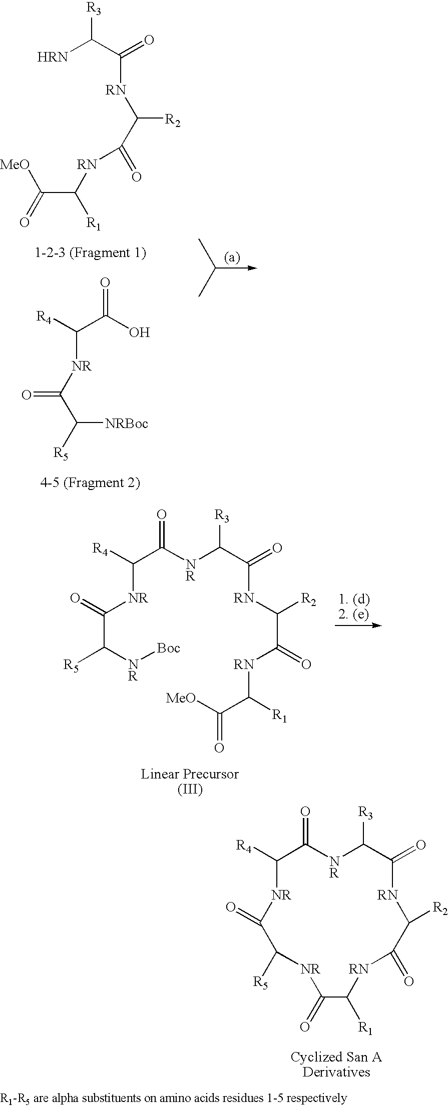

Conditions: a) coupling agent*, DIPEA (3 equiv), CH₂Cl₂ (0.1M), b)TFA (20%), Anisole (2 equiv), CH₂Cl₂, c) LiOH (4 equiv), MeOH d) HCl in THF (0.05M), Anisole (2 equiv), e) HATU (0.7 equiv), DEPBT (0.7 equiv), TBTU (0.7 equiv), DIPEA (6 equiv). THF:CH₃CN:DCM (2:2:1) 0.007M. *TBTU (1.2 equiv), and/or HATU (0.75 equiv).

Peptide derivatives of formula (I) may also be prepared using approaches familiar to those skilled in the ait of peptide chemistry or simple modifications of those approaches. For example, while Scheme 1 exemplifies the use of Boc-protecting group, one of skill in the art would readily appreciate that other protecting group strategies could be employed to provide fully deprotected linear pentapeptides which represent the penultimate cyclization precursors.

Using the route outlined in Scheme 1, three subsets of San A derivatives were prepared, as further described herein. The first subset comprises only L-amino acids; non-limiting examples are shown in FIG. 1. The second subset comprises only D-amino acids; non-limiting examples are shown in FIG. 2. The third subset comprises both L- and D-amino acids. Non-limiting examples comprising a single D-amino acid are shown in FIG. 3. Non-limiting examples comprising two or more D-amino acids are shown in FIG. 4. Additional compounds of the invention are shown in FIG. 5.

Synthesis of Cyclic Pentapeptoids of Formula (II)

Linear peptides often have problems with solubility, and degradation within cells. Macrocyclic peptides show improved cellular stability over linear peptides, but often have difficulty being soluble at an appropriate level needed for a commercial drug. A recent solution to these two problems is the use of peptidomimetics, known as “peptoids”, are peptide-like compounds where functionality is located on the amide nitrogen rather than on the alpha carbon of the amino acid. Such compounds, which formally comprise N-alkylated glycine residues, have improved solubility and stability within cells. Peptoids of the invention may be prepared as shown in Scheme 2.

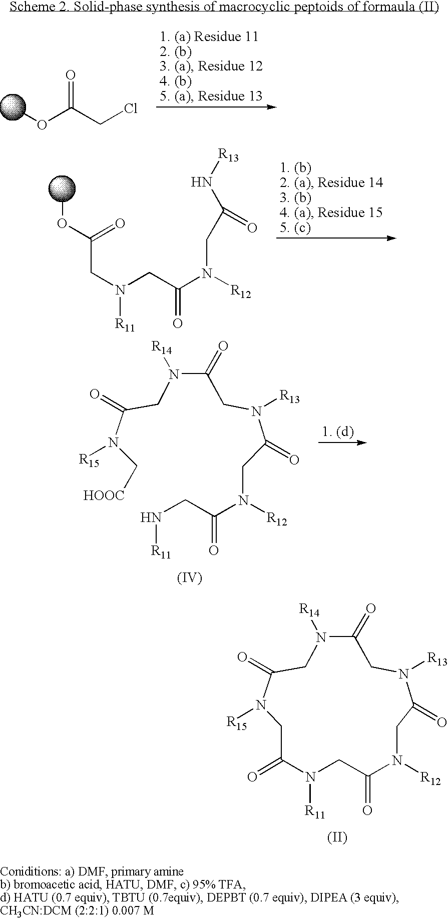

Peptoids are synthesized using the Zuckermann and Moos method where the synthesis of each N-substituted glycine is built in two steps. (Zuckermann, et al., J. Am. Chem. Soc. 1992, 114,10646-10647). The first involves an acylation step and the second step involves a nucleophilic displacement. Starting with a Wang resin and a halo acid attached to the resin, coupling of residues 11 in parallel provides the first N-substituted glycine. Coupling of bromoacetic acid using HATU (known to be superior for coupling secondary amines to acids), and subsequent coupling of the second amine (residue 12) provides the second N-substituted glycine. Repeating with this process using amines 13, 14, and 15 (corresponding to residues 13, 14, and 15) gives the linear pentapeptoid. Cleavage from the resin and subsequent cyclization using the standard macrocyclization conditions given for compounds of formula (I) provides the desired peptoids. All compounds are purified via reverse phase HPLC, and fully characterized using NMR, LCMS and high resolution Mass Spectrometry.

In the present invention, cyclic pentapeptoids are designed to “match” the active structures identified in the cyclic pentapeptide series. Peptoid derivatives of formula (II) may be prepared as shown in Scheme 2. These cyclic pentapeptoid compounds allow examination of the impact of chirality, the positioning of the sidechain relative to the amide carbonyl, the necessity for aromatic versus alkyl, and/or polar versus non-polar side chains, and the relevance of size or “fit”.

### Structural Studies

Compounds of the invention have been further characterized by a variety of structural techniques. The solution conformation of potent compounds, have been compared to the structures to relatively non-potent compounds, including for example, San A, utilizing HSQC, TOCSEY, NOESY and ROESY NMR. Such NOE and ROE values may be used in conjunction with molecular modeling to determine the lowest energy conformation of these compounds. CoMFA models were generated using the NMR data and HCT116, HCT15 and PL45 screening data sets. The CoMFA models were trained using the natural logarithmic quantity log [inh. %]. CoMFA modeling provides a projection of a pharmacophore map that is generated by fitting the experimental inhibition data using a partial least squares fit. The pharmacophore maps for each cell line were almost identical, indicating the compounds were most likely presenting the amino acid side chains in the same conformation in each cell line.

Solid-state structures for compounds of the invention are determined using small molecule X-ray crystallization.

### EXAMPLES

The following examples describe exemplary compounds of the invention, processes for their preparation, and pharmaceutical compositions made by these processes. The examples also describe assays used to characterize the compounds of the invention. These examples are offered to illustrate but not to limit the invention. The principle features of the invention can be employed in various embodiments without departing from the scope of the invention. Various modifications may be made by the skilled person without departing from the true spirit and scope of the invention.

### Example 1

### Synthesis of Cyclic Pentapeptides of Formula (I)

### General Remarks

All coupling reactions were performed under argon atmosphere with the exclusion of moisture. All reagents were used as received. Anhydrous methylene chloride Dri Solv (EM) and anhydrous acetonitrile Dri Solv (EM) were bought from VWR, and were packed under nitrogen with a septum cap. Diisopropylethylamine (DIPEA) was purchased from Aldrich, packaged under nitrogen in a Sure Seal bottle. The coupling agents HATU and PyAOP were purchased fiom Perspective:Applied Biosystems at Lincoln Center Dr. Foster City, Calif. 94404. The coupling agents 2(1-H-benzotriazole- 1-yl)-1,1,3-tetramethyl-uronium tetrafluoroborate (TBTU) and PyBROP were purchased from NovaBiochem. DEPBT [3(diethoxyphosphoryloxy)-1,2,3-benzotriazone-4(3H)] was purchased from Aldrich.

¹H NMR spectra were recorded on a Varian at 500 MHz, typically in CD₃OD using an internal TMS standard. LCMS data were obtained using a HP 1100 Finnigan LCQ. Flash chromatography was performed on 230-400 mesh 32-74 micron 60 angstrom silica gel from Bodman Industries.

### General Peptide Synthesis

All peptide coupling reactions were carried out under argon with dried solvent, using methylene chloride for dipeptide and tripeptide couplings and acetonitrile for all other peptide couplings. The amine (1.1 equivalents) and acid (1 equivalent) were weighed into a dry flask along with 3 equivalents of DIPEA and 1.1 equivalents of TBTU.* Anhydrous methylene chloride was added for a 0.1 M solution. The solution was stirred at room temperature and reactions were monitored by TLC. Reactions were run for 4-24 hours before working up by washing with saturated aqueous ammonium chloride. (Note: it acetonitrile was used for the reaction, methylene chloride was added to the reaction upon workup and then the resulting solution was washed with ammonium chloride). After back extraction of aqueous layers with methylene chloride, organic layers were combined, dried over sodium suylfate, filtered and concentrated. Flash chromatography using 0-100% ethylacetate-hexame gave the desired peptides.

*Some coupling reactions would not go to completion using only TBTU and therefore HATU, and/or DEPBT were used. In certain cases, 1.1 equivalents of all three coupling reagents were used.

### General Amine Deprotection

Amines were deprotected using 20% TFA in methylene chloride (0.1M) with two equivalents of anisole. The reactions were monitored by TLC, where the TLC sample was first worked up in a mini-workup using deionized (DI) water and methylene chloride to remove TFA. Reactions were allowed to run for 1-2 hours and then concentrated in vacuo.

### General Acid Deprotection

Acids were deprotected using 4 equivalents of lithium hydroxide (or until pH ˜11) in methanol (0.1M). The peptide was placed in a flask, along with lithium hydroxide and methanol and stirred overnight. Within 21 hours the acid was usually deprotected. Work-up of reactions involved the acidification of the reaction solution using HCL to pH=1. The aqueous solution was extracted three times with methylene chloride, and the combined organic layer was dried, filtered and concentrated in vacuo.

### Macrocyclization Procedure (In Situ)

All pentapeptides were acid and amine deprotected using HCL (8 drops per 0.3 mmols of linear pentapeptide) in THF (0.05M). Addition of anisole (2 equivalents) was added to the reaction and the reaction was stirred at room temperature. The reaction typically took 4 days, although it was checked after 24 hours via LCMS and TLC. The LCMS typically indicated the reaction was ˜50% complete after the first day. An additional four drops of HCl per 0.3 mmols of pentapeptide were added, and stirring at room temperature was continued overnight; checking the reaction via LCMS showed ˜75% completion. An additional 2 drops of HCl per 0.3 mmols of pentapeptide were added on the third day. On the fourth day, verification of the presence of the free amine and free acid and disappearance of the starting linear protected pentapeptide permitted workup. The reaction was concentrated in vacuo and the crude, dry, doubly deprotected peptide was dissolved in a minimum solution of THF:acetonitrile:methylene chloride (2:2:1 ratio).

For the in situ macrocyclization, three coupling agents were used: DEPBT, HATU, and TBTU (˜0.5 to 0.75 equivalents each). The coupling agents were dissolved in a calculated volume of dry 40% THF, 40% acetonitrile and 20% methylene chloride that would give a 0.007 M solution when included in the volume used for the fully deprotected peptide. (A mixture of solvents was used to facilitate dissolution of the coupling reagents.) The coupling agents were then added to the deprotected peptide solution. DIPEA (6 equivalents or more, in order to neutralize the pH) were then added to the reaction. After 1 hour, TLC and LCMS showed a distinct product spot. (Analytical samples were worked up prior to LCMS analysis.) The reactions were usually complete within 2-6 hours, and monitoring the disappearance of the fully deprotected pentapeptide via LCMS was the easiest method of determining completion. Upon completion, the reaction was worked up by washing with aqueous ammonium chloride. After back extraction of the aqueous layers with large quantities of methylene chloride, the organic layers were combined, dried over sodium sulfate, filtered and concentrated. All macrocycles were purified by initially running the crude compound through a silica gel plug with ethyl acetate/hexane solvent system, then running a column on the isolated product. Finally, if necessary, reverse phase HPLC was used for additional purification using a gradient of acetonitrile and DI water with 0.1% TFA. The final products were isolated in yields ranging from 23% to 90% depending on the substrate.

### Results

Using the procedures outlined above, the tripeptide fragment (Scheme 1), comprising residues 1-3, was synthesized as follows. Residue 1 was coupled to the N-Boc protected residue 2 to give the Boc-protected 1-2 dipeptide in 80-94% yield. Deprotection of the amine on residue 2 using TFA gave the free dipeptide amines in quantitative yields. Coupling of the dipeptide to residue 3 gave the desired tripeptide in good yields (80%-95%). The synthesis of dipeptide fragment comprising residues 4-5, was synthesized as follows. Residue 4 was coupled to Boc-protected residue 5 to give the Boc-protected 4-5 dipeptide in 90-95% yield. The amine was deprotected on tripeptide Fragment 1 using TFA and the acid was deprotected in dipeptide Fragment 2 using lithium hydroxide. Fragment 1 and Fragment 2 were coupled using multiple coupling agents yielding linear pentapeptides in 66-90% yields.

All compounds of the invention gave satisfactory LCMS and ¹H NMR spectral data.

Using the macrocyclization procedure above, the following cyclic pentapeptides were isolated, and their chemical yields obtained: Compound 1 (65%); t_{R} 6.93, m/z=588.0, m/z=1171.8 (MW 586); Compound 22 (39%); t_{R} 6.63, m/z=586.8, m/z=1171.7 (MW 586); Compound 23 (85%); t_{R} 6.15, m/z=586.7, m/z=1171.8 (MW 586); Compound 24 (65%); t_{R} 6.93, m/z=586.8, m/z=1171.8 (MW 586); Compound 25 (53%); t_{R} 7.02, m/z=587.1, m/z=1171.8 (MW 586); Compound 26 (37%); t_{R} 7.05, m/z=586.8, m/z=1171.9 (MW 586); Compound 32 (81%); t_{R} 7.00, m/z=600.7; m/z=1199.2 (MW 600); Compound 38 (40%); t_{R} 6.40, m/z=601.0, m/z=1221.6 (MW 600); Compound 46 (35%); t_{R} 6.49, m/z=586.8, m/z=1171.6 (MW 586).

Other compounds of the invention were prepared using the general routes described above, to provide the compounds indicated in Table 1, below. Standard three letter codes are used to identify amino acid residues. Non-natural amino acids are identified in the sequence as Xaa, and the specific non-natural amino acid is indicated in the column corresponding to that residue number in the cyclic pentapeptide backbone. Thus, for example, compound 3 has the sequence cyclo[-Phe-Xaa-Val-Leu-Leu-], where Phe corresponds to residue 1, and the residue in position 2 (i.e., Xaa-2) is NMeLeu.

TABLE 1
Compounds of formula (I).

| | | | | | | | |
|---|---|---|---|---|---|---|---|
| Cpd | SEQ ID | | | | | | |
| # | NO: | Sequence | Xaa-1 | Xaa-2 | Xaa-3 | Xaa-4 | Xaa-5 |

| | | | | | | | |
|---|---|---|---|---|---|---|---|
| 1 | SEQ ID | cyclo[-Phe-Leu-Val- | | | | | |
| | NO: 1 | Leu-Leu-] | | | | | |
| 2 | SEQ ID | cyclo[-Xaa-Xaa-Xaa- | DPhe | DLeu | DVal | DLeu | DLeu |
| | NO: 18 | Xaa-Xaa-] | | | | | |
| 3 | SEQ ID | cyclo[-Phe-Xaa-Val- | | NMeLeu | | | |
| | NO: 2 | Leu-Leu-] | | | | | |
| 4 | SEQ ID | cyclo[-Xaa-Xaa-Xaa- | DPhe | DNMeLeu | DVal | DLeu | DLeu |
| | NO: 18 | Xaa-Xaa-] | | | | | |
| 5 | SEQ ID | cyclo[-Phe-Xaa-Val- | | NMeLeu | | | NMeLeu |
| | NO: 3 | Leu-Xaa-] | | | | | |
| 6 | SEQ ID | cyclo[-Xaa-Xaa-Xaa- | DPhe | DNMeLeu | DVal | DLeu | DNMeLeu |
| | NO: 18 | Xaa-Xaa-] | | | | | |
| 7 | SEQ ID | cyclo[-Phe-Leu-Xaa- | | | NMeVal | | |
| | NO: 4 | Leu-Leu-] | | | | | |
| 8 | SEQ ID | cyclo[-Xaa-Xaa-Xaa- | DPhe | DLeu | DNMeVal | DLeu | DLeu |
| | NO: 18 | Xaa-Xaa-] | | | | | |
| 9 | SEQ ID | cyclo[-Phe-Leu-Val- | | | | | NMeLeu |
| | NO: 5 | Leu-Xaa-] | | | | | |
| 11 | SEQ ID | cyclo[-Phe-Leu-Xaa- | | | NMeVal | | NMeLeu |
| | NO: 6 | Leu-Xaa-] | | | | | |
| 12 | SEQ ID | cyclo[-Xaa-Xaa-Xaa- | DPhe | DLeu | DNMeVal | DLeu | DNMeLeu |
| | NO: 18 | Xaa-Xaa-] | | | | | |
| 13 | SEQ ID | cyclo[-Phe-Xaa-Xaa- | | NMeLeu | NMeVal | | NMeLeu |
| | NO: 7 | Leu-Xaa-] | | | | | |
| 14 | SEQ ID | cyclo[-Xaa-Xaa-Xaa- | DPhe | DNMeLeu | DNMeVal | DLeu | DNMeLeu |
| | NO: 18 | Xaa-Xaa-] | | | | | |
| 15 | SEQ ID | cyclo[-Phe-Xaa-Xaa- | | NMeLeu | NMeVal | | |
| | NO: 8 | Leu-Leu-] | | | | | |
| 16 | SEQ ID | cyclo[-Phe-Gly-Val- | | | | | |
| | NO: 9 | Leu-Leu-] | | | | | |
| 17 | SEQ ID | cyclo[-Phe-Xaa-Val- | | Sar | | | |
| | NO: 2 | Leu-Leu-] | | | | | |
| 18 | SEQ ID | cyclo[-Phe-Leu-Ala- | | | | | |
| | NO: 10 | Leu-Leu-] | | | | | |
| 19 | SEQ ID | cyclo[-Phe-Leu-Xaa- | | | (αEt)Gly | | |
| | NO: 4 | Leu-Leu-] | | | | | |
| 20 | SEQ ID | cyclo[-Phe-Ile-Val- | | | | | |
| | NO: 11 | Leu-Leu-] | | | | | |
| 21 | SEQ ID | cyclo[-Phe-Ala-Val- | | | | | |
| | NO: 12 | Leu-Leu-] | | | | | |
| 22 | SEQ ID | cyclo[-Xaa-Leu-Val- | DPhe | | | | |
| | NO: 13 | Leu-Leu-] | | | | | |
| 23 | SEQ ID | cyclo[-Phe-Xaa-Val- | | DLeu | | | |
| | NO: 2 | Leu-Leu-] | | | | | |
| 24 | SEQ ID | cyclo[-Phe-Leu-Xaa- | | | DVal | | |
| | NO: 4 | Leu-Leu-] | | | | | |
| 25 | SEQ ID | cyclo[-Phe-Leu-Val- | | | | DLeu | |
| | NO: 17 | Xaa-Leu-] | | | | | |
| 26 | SEQ ID | cyclo[-Xaa-Xaa-Val- | DPhe | DLeu | | | |
| | NO: 25 | Leu-Leu-] | | | | | |
| 27 | SEQ ID | cyclo[-Xaa-Xaa-Xaa- | DPhe | DLeu | DVal | | |
| | NO: 26 | Leu-Leu-] | | | | | |
| 28 | SEQ ID | cyclo[-Xaa-Xaa-Xaa- | DPhe | DLeu | DVal | DLeu | |
| | NO: 27 | Xaa-Leu-] | | | | | |
| 29 | SEQ ID | cyclo[-Xaa-Leu-Val- | NMePhe | | | | |
| | NO: 13 | Leu-Leu-] | | | | | |
| 30 | SEQ ID | cyclo[-Xaa-Leu-Xaa- | NMePhe | | NMeVal | | |
| | NO: 14 | Leu-Leu-] | | | | | |
| 31 | SEQ ID | cyclo[-Phe-Leu-Xaa- | | | DNMeVal | | |
| | NO: 4 | Leu-Leu-] | | | | | |
| 32 | SEQ ID | cyclo[-Phe-Xaa-Val- | | DNMeLeu | | | |
| | NO: 2 | Leu-Leu-] | | | | | |
| 33 | SEQ ID | cyclo[-Phe-Leu-Val- | | | | | DLeu |
| | NO: 5 | Leu-Xaa-] | | | | | |
| 34 | SEQ ID | cyclo[-Phe-Leu-Val- | | | | NMeLeu | |
| | NO: 17 | Xaa-Leu-] | | | | | |
| 35 | SEQ ID | cyclo[-Phe-Leu-Val- | | | | | |
| | NO: 15 | Ile-Leu-] | | | | | |
| 36 | SEQ ID | cyclo[-Phe-Val-Leu- | | | | | |
| | NO: 16 | Leu-Val-] | | | | | |
| 37 | SEQ ID | cyclo[-Phe-Xaa-Val- | | nBug | | | |
| | NO: 2 | Leu-Leu-] | | | | | |
| 38 | SEQ ID | cyclo[-Phe-Leu-Val- | | | | NMeLeu | DLeu |
| | NO: 19 | Xaa-Xaa-] | | | | | |
| 39 | SEQ ID | cyclo[-Xaa-Leu-Val- | DPhe | | | NMeLeu | DLeu |
| | NO: 30 | Xaa-Xaa-] | | | | | |
| 40 | SEQ ID | cyclo[-Xaa-Leu-Val- | Tic | | | | |
| | NO: 13 | Leu-Leu-] | | | | | |
| 42 | SEQ ID | cyclo[-Phe-Leu-Xaa- | | | DLeu | | |
| | NO: 4 | Leu-Leu-] | | | | | |
| 43 | SEQ ID | cyclo[-Xaa-Val-Xaa- | DPhe | | DLeu | DLeu | |
| | NO: 28 | Xaa-Ile-] | | | | | |
| 46 | SEQ ID | cyclo[-Phe-Leu-Val- | | | | DLeu | DLeu |
| | NO: 19 | Xaa-Xaa-] | | | | | |
| 47 | SEQ ID | cyclo[-Phe-Leu-Xaa- | | | DVal | DLeu | DLeu |
| | NO: 29 | Xaa-Xaa-] | | | | | |
| 48 | SEQ ID | cyclo[-Phe-Xaa-Xaa- | | DNMeLeu | DNMeVal | | |
| | NO: 8 | Leu-Leu | | | | | |
| 49 | SEQ ID | cyclo[-Phe-Val-Xaa- | | | DVal | | Sar |
| | NO: 20 | Leu-Xaa-] | | | | | |
| 50 | SEQ ID | cyclo[-Phe-Leu-Xaa- | | | DNMeVal | Cha | |
| | NO: 21 | Xaa-Leu-] | | | | | |
| 51 | SEQ ID | cyclo[-Xaa-Leu-Val- | DTyr | | | | |
| | NO: 13 | Leu-Leu-] | | | | | |
| 52 | SEQ ID | cyclo[-Xaa-Leu-Val- | DTrp | | | | |
| | NO: 13 | Leu-Leu-] | | | | | |
| 53 | SEQ ID | cyclo[-Phe-Leu-Xaa- | | | DSer | | |
| | NO: 4 | Leu-Leu-] | | | | | |
| 54 | SEQ ID | cyclo[-Phe-Leu-Val- | | | | | DNMeLeu |
| | NO: 5 | Leu-Xaa-] | | | | | |
| 55 | SEQ ID | cyclo[-Phe-Xaa-Val- | | DNMePhe | | | |
| | NO: 2 | Leu-Leu-] | | | | | |
| 56 | SEQ ID | cyclo[-Phe-Xaa-Val- | | DPhe | | | |
| | NO: 2 | Leu-Leu-] | | | | | |
| 57 | SEQ ID | cyclo[-Phe-Val-Xaa- | | | DVal | | |
| | NO: 22 | Val-Val-] | | | | | |
| 58 | SEQ ID | cyclo[-Phe-Val-Xaa- | | | D(αEt)Gly | | |
| | NO 22 | Val-Val-] | | | | | |
| 59 | SEQ ID | cyclo[-Phe-Val-Xaa- | | | DSer | | |
| | NO: 22 | Val-Val-] | | | | | |
| 60 | SEQ ID | cyclo[-Phe-Xaa-Val- | | DSer | | | |
| | NO: 23 | Val-Val-] | | | | | |
| 61 | SEQ ID | cyclo[-Phe-Leu-Xaa- | | | DPhe | | |
| | NO: 4 | Leu-Leu-] | | | | | |
| 63 | SEQ ID | cyclo[-Phe-Xaa-Val- | | DSer | | | |
| | NO: 2 | Leu-Leu-] | | | | | |
| 64 | SEQ ID | cyclo[-Phe-Val-Xaa- | | | DNMeVal | DSer | |
| | NO: 24 | Xaa-Leu-] | | | | | |
| 65 | SEQ ID | cyclo[-Phe-Xaa-Xaa- | | DLeu | (αEt)Gly | | |
| | NO: 8 | Leu-Leu-] | | | | | |
| 66 | SEQ ID | cyclo[-Phe-Leu-Xaa- | | | D(αEt)Gly | | |
| | NO: 4 | Leu-Leu-] | | | | | |
| 67 | SEQ ID | cyclo[-Phe-Xaa-Xaa- | | DLeu | DVal | | |
| | NO: 8 | Leu-Leu-] | | | | | |
| 68 | SEQ ID | cyclo[-Phe-Leu-Xaa- | | | DNMeVal | | DLeu |
| | NO: 6 | Leu-Xaa-] | | | | | |
| 69 | SEQ ID | cyclo[-Tyr-Leu-Xaa- | | | DNMeVal | | |
| | NO: 31 | Leu-Leu-] | | | | | |
| 70 | SEQ ID | cyclo[-Val-Leu-Xaa- | | | DNMeVal | | |
| | NO: 32 | Leu-Leu-] | | | | | |
| 71 | SEQ ID | cyclo[-Phe-Leu-Xaa- | | | DNMeVal | | NMeLeu |
| | NO: 6 | Leu-Xaa-] | | | | | |
| 72 | SEQ ID | cyclo[-Phe-Leu-Xaa- | | | DVal | | DLeu |
| | NO: 6 | Leu-Xaa-] | | | | | |
| 73 | SEQ ID | cyclo[-Phe-Leu-Xaa- | | | DVal | | NMeLeu |
| | NO: 6 | Leu-Xaa-] | | | | | |
| 74 | SEQ ID | cyclo[-Xaa-Leu-Val- | DpBrPhe | | | | |
| | NO: 13 | Leu-Leu-] | | | | | |
| 75 | SEQ ID | cyclo[-Xaa-Leu-Xaa- | DPhe | | DNMeVal | | |
| | NO: 14 | Leu-Leu-] | | | | | |
| 76 | SEQ ID | cyclo[-Xaa-Xaa-Xaa- | DPhe | DLeu | DNMeVal | | |
| | NO: 26 | Leu-Leu-] | | | | | |
| 77 | SEQ ID | cyclo[-Xaa-Leu-Xaa- | DPhe | | DVal | | |
| | NO: 14 | Leu-Leu-] | | | | | |
| 78 | SEQ ID | cyclo[-Xaa-Leu-Xaa- | DPhe | | DLys | | |
| | NO: 14 | Leu-Leu-] | | | | | |
| 79 | SEQ ID | cyclo[-Phe-Leu-Xaa- | | | DVal | | |
| | NO: 33 | Lys-Leu-] | | | | | |
| 80 | SEQ ID | cyclo[-Xaa-Xaa-Xaa- | DPhe | DLeu | DVal | | |
| | NO: 34 | Lys-Leu-] | | | | | |
| 81 | SEQ ID | cyclo[-Phe-Leu-Val- | | | | | |
| | NO: 35 | Lys-Leu-] | | | | | |
| 82 | SEQ ID | cyclo[-Phe-Leu-Xaa- | | | D(αEt)Gly | Cha | |
| | NO: 21 | Xaa-Leu-] | | | | | |
| 83 | SEQ ID | cyclo[-Phe-Leu-Xaa- | | | DNMeVal | | |
| | NO: 36 | Leu-Lys-] | | | | | |
| 84 | SEQ ID | cyclo[-Phe-Leu-Xaa- | | | DLys | | |
| | NO: 4 | Leu-Leu-] | | | | | |
| 85 | SEQ ID | cyclo[-Xaa-Leu-Val- | DTyr | | | | |
| | NO: 37 | Lys-Leu-] | | | | | |
| 86 | SEQ ID | cyclo[-Xaa-Leu-Val- | DTrp | | | | |
| | NO: 38 | Arg-Leu-] | | | | | |
| 87 | SEQ ID | cyclo[-Lys-Leu-Val- | | | | | |
| | NO: 39 | Leu-Leu-] | | | | | |
| 88 | SEQ ID | cyclo[-Xaa-Xaa-Xaa- | DPhe | DSer | DLys | | |
| | NO: 26 | Leu-Leu-] | | | | | |
| 89 | SEQ ID | cyclo[-Lys-Leu-Xaa- | | | DVal | | |
| | NO: 40 | Leu-Leu-] | | | | | |
| 90 | SEQ ID | cyclo[-Xaa-Xaa-Xaa- | DLys | DLeu | DVal | | |
| | NO: 26 | Leu-Leu-] | | | | | |
| 91 | SEQ ID | cyclo[-Phe-Val-Xaa- | | | D(OBzl) | | |
| | NO: 22 | Val-Val-] | | | Ser | | |
| 92 | SEQ ID | cyclo[-Phe-Xaa-Val- | | D(OBzl) | | | |
| | NO: 23 | Val-Val-] | | Ser | | | |
| 93 | SEQ ID | cyclo[-Phe-Leu-Xaa- | | | DVal | CBZLys | |
| | NO: 21 | Xaa-Leu-] | | | | | |
| 94 | SEQ ID | cyclo[-Xaa-Xaa-Xaa- | DPhe | DLeu | DVal | CBzLys | |
| | NO: 27 | Xaa-Leu-] | | | | | |
| 95 | SEQ ID | cyclo[-Phe-Leu-Val- | | | | CBZLys | |
| | NO: 17 | Xaa-Leu-] | | | | | |
| 100 | SEQ ID | cyclo[-Xaa-Xaa-Xaa- | DPhe | DLeu | DVal | | |
| | NO: 4 | Leu-Ser-] | | | | | |
| 101 | SEQ ID | cyclo[-Xaa-Xaa-Xaa- | DPhe | DLeu | DVal | | |
| | NO: 42 | Leu-Lys-] | | | | | |
| 102 | SEQ ID | cyclo[-Phe-Xaa-Val- | | DNMePhe | | | |
| | NO: 43 | Leu-Ser-] | | | | | |
| 103 | SEQ ID | cyclo[-Phe-Xaa-Val- | | DNMePhe | | | |
| | NO: 44 | Leu-Lys-] | | | | | |
| 104 | SEQ ID | cyclo[-Phe-Xaa-Val- | | DNMePhe | | Cha | |
| | NO: 45 | Xaa-Ser-] | | | | | |
| 105 | SEQ ID | cyclo[-Phe-Xaa-Xaa- | | DNMePhe | DVal | Cha | |
| | NO: 46 | Xaa-Ser-] | | | | | |
| 107 | SEQ ID | cyclo[-Phe-Xaa-Val- | | DNMePhe | | CBZLys | |
| | NO: 47 | Xaa-Leu-] | | | | | |
| 108 | SEQ ID | cyclo[-Phe-Xaa-Val- | | DNMePhe | | | |
| | NO: 48 | Lys-Leu-] | | | | | |
| 110 | SEQ ID | cyclo[-Xaa-Xaa-Val- | DTyr | DNMePhe | | | |
| | NO: 25 | Leu-Leu-] | | | | | |
| 111 | SEQ ID | cyclo[-Phe-Leu-Xaa- | | | DNMeVal | DLeu | |
| | NO: 21 | Xaa-Leu-] | | | | | |
| 112 | SEQ ID | cyclo[-Phe-Leu-Xaa- | | | DNMeVal | | CBZLys |
| | NO: 6 | Leu-Xaa-] | | | | | |
| 113 | SEQ ID | cyclo[-Phe-Xaa-Val- | | DNMePhe | | Cha | (OBzl)Ser |
| | NO: 49 | Xaa-Xaa-] | | | | | |
| 114 | SEQ ID | cyclo[-Phe-Xaa-Xaa- | | DNMePhe | DVal | Cha | (OBzl)Ser |
| | NO: 50 | Xaa-Xaa-] | | | | | |
| 115 | SEQ ID | cyclo[-Xaa-Leu-Val- | DNMePhe | | | | DTyr |
| | NO: 51 | Leu-Xaa-] | | | | | |
| 116 | SEQ ID | cyclo[-Xaa-Leu-Val- | DNMeTyr | | | | |
| | NO: 13 | Leu-Leu-] | | | | | |
| 117 | SEQ ID | cyclo[-Xaa-Leu-Xaa- | DNMeTyr | | DVal | | |
| | NO: 14 | Leu-Leu-] | | | | | |
| 118 | SEQ ID | cyclo[-Xaa-Xaa-Val- | DTyr | DNMePhe | | NMeLeu | |
| | NO: 52 | Xaa-Leu-] | | | | | |
| 119 | SEQ ID | cyclo[-Xaa-Xaa-Val- | DTyr | DNMePhe | | NMeLeu | DLeu |
| | NO: 53 | Xaa-Xaa-] | | | | | |

### Example 2

### Synthesis of Cyclic Peptoid Derivatives of Formula (II)

Macrocyclic peptoids are synthesized using the Zuckermann and Moos method where the synthesis of each N-substituted glycine is built in two steps. A Wang resin is acylated with an alpha-halo acid, and the halo substituent is displaced by coupling of the amines corresponding to residue 11, in parallel, to provide the first N-substituted glycine. Coupling of bromoacetic acid using HATU in DMF, and subsequent coupling of the second amine monomer (residue 12) provides the second N-substituted glycine. Repeating the process using amines 13, 14, and 15 (corresponding to residues 13, 14, and 15) gives the linear pentapeptoid. Cleavage from the resin with 95% TFA and subsequent macrocyclization using HATU, TBTU and DEPBT (0.7 equivalents of each), DIPEA (3 equivalents) in a 2:2:1 mixutre of acetonitrile, THF and dichloromethane at a dilution of 0.007M, as described for compounds of formula (I), provides the desired cyclic pentapeptoids. All compounds are purified via reverse phase HPLC, and fully characterized using NMR, LCMS and high resolution Mass Spectrometry.

### Example 3

### ³H-Thymidine Incorporation Assay

### Methods:

³H-thymidine incorporation assays involved culturing cells in 96 well plates at a concentration of 3000 cells/well. After incubation for approximately 16 hours, the media was replaced with fresh media both with and without the addition of compounds, to give 1% DMSO in wells. Cells were then incubated for 56 hours, whereupon ³H-thymidine was added for 16 hours (thus cells were incubated with compound for a total of 72 hours). Cells were then washed, fixed, solubilized and the DNA was isolated and counted in a scintillation counter using standard approaches. Decreases in ³H-thymidine incorporation, as compared to the control (control was cells with 1% DMSO and no compound), indicated cells were no longer progressing through the cell cycle and synthesizing DNA, thus providing the % growth inhibition. Each assay will be performed with triplicate wells and each experiment will be repeated 3 times so that accurate estimates of the IC₅₀ can be obtained.

### Example 4

### Annexin V Apoptosis Assay

### Methods:

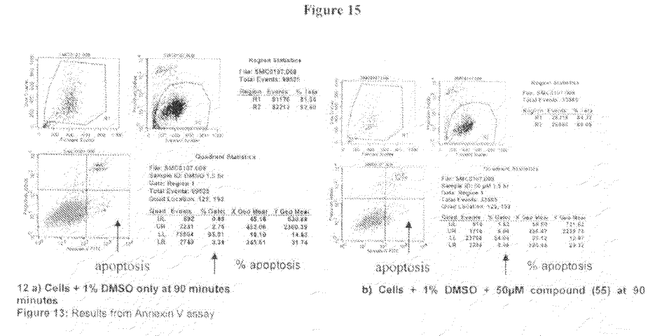

Following the standard protocol, PL-45 pancreatic cancer cells were seeded in tissue culture plates in media. The plates were incubated in CO₂ for 6 hours at 37° C. to allow cells to attach to the plate. The serum containing media was removed, and fresh serum and media were added. The cells were then incubated for 24 hours. Next, 50 μM compound (55) was added to the cells, which were incubated for two time points: 1.5 hours and 3 hours (additional time points and concentrations are planned, but for purposes of gathering preliminary data these were chosen). Two control experiments were run at each time point (cells with no DMSO), and cells with 1% DMSO but no compound. The cells were then rinsed with binding buffer, the buffer was removed, and the cells were resuspended. Annexin V and propidium iodide (PI) were added to the cells. Three controls were run: annexin V only, PI only, and both annexin V and PI to mixtures of the cells from all six experiments. The six experiments involved three experiments at each time point: a) cells only, b) cells+1% DMSO, c) cells+1% DMSO+compound and 3 hour experiments included the same three experiments. All nine experiments were then analyzed by flow cytometry and the results are shown in FIGS. 15(a) and 15(b). It is impoltant to note that both a) cells and b) cells+1% DMSO gave the same results, indicating DMSO had no impact on apoptosis.

### Example 5

### Identification of San A Targets.

### Methods:

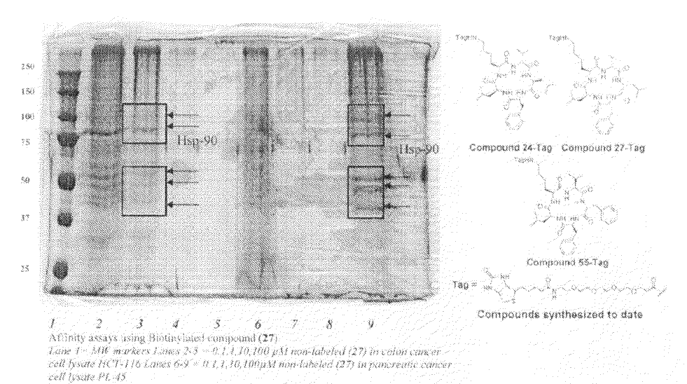

In order to identify cellular proteins that bind to the San A analogs and may mediate the cytotoxicity of these drugs, 3 biotin-containing compounds were synthesized: (24-Biotin), (27-Biotin), and (55-Biotin) (FIG. 14). These 3 compounds were chosen as they have potent activity against both the colon and pancreatic cell lines.

Biotinylated compounds (24-Biotin) and (27-Biotin) were used in affinity purification assays to isolate proteins from HCT-116 and PL-45 cells that bind to these compounds. After incubation of the cell lysates with the biotinylated compounds, streptavidin resin was added and the unbound proteins were removed by washing 5 times with buffer. Re-suspension of the resin in buffer followed by incubation of the resin-bound (27-Biotin)-protein complex with non-biotinylated (27) allowed for competitive elution of protein targets from the resin (27-Biotin).

### Results:

Analysis of the eluted proteins by SDS page demonstrated 5 bands whose intensity increased in proportion to the concentration of the biotinylated compound (FIG. 14). Both compounds (24-Biotin) and (27-Biotin) pulled out the same 5 protein bands. The identity of these proteins was established using MS/MS fingerprinting software. Four of the bands were proteins typically pulled out due to hydrophobic interactions: mysoin, alpha and beta tubulin, and actin. However, one band was identified as Hsp90-beta. and the excellent expectancy value of Hsp90 C-terminus indicates a high likelihood that it binds to the San A derivatives.

Using (24-Biotin) and (27-Biotin), the same proteins were recovered in both HCT-116 and PL-45 cells, suggesting that the compounds of the invention act via the same mechanism in colon and pancreatic cancer cell lines.

### Example 6

### Fluorescence Studies with San A Analogs

### Methods:

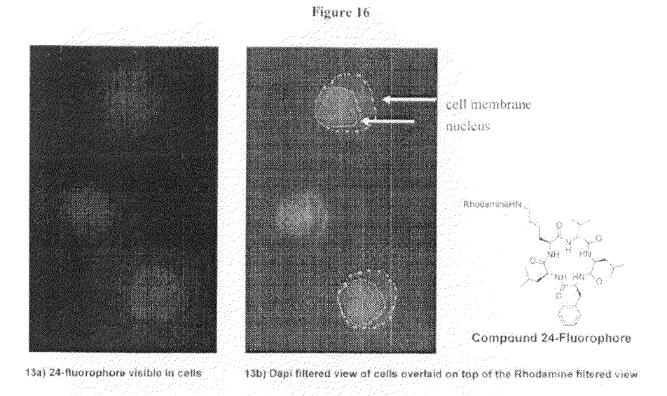

To determine where compounds of the invention accumulate in the cell, fluorochrome-labeled analogs of compounds (1), (24), and (55) were synthesized with rhodamine attached at position 4. These compounds were made using the same synthetic strategy utilized for the Biotinylated compounds described in FIG. 14. A lysine was incorporated at residue 4, whereupon Rhodamine was attached to the lysine after completion of the compound synthesis (FIG. 16). PL-45 cells were incubated with a 5 μM concentration of (24)-Fluorophore and the intracellular distribution as a function of time was documented using microscopy. Dapi was also used in order to stain the nucleus. Images using the Rhodamine filter and the Dapi filter were taken of the same cells, and then over-laid (FIG. 16(b)).

### Results:

The red images shown in FIG. 16(a) that the compounds, which are covalently bound to Rhodamine, penetrate the cell membrane and enter the cytosol.

1. A compound of formula (I): 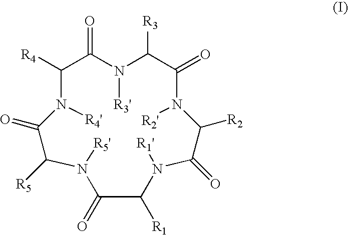
or a pharmaceutically acceptable salt or hydrate form thereof; and including any stereoisomers thereof;
wherein each of R_{1′}, R_{2′}, R_{3′}, R_{4′}, and R_{5′} independently represents H, or C1-C4 alkyl; and wherein R_{1′} may cyclize with R₁ to form a 5-10 membered azacyclic ring;
R₁ represents a C1-C4 alkyl, C5-C12 arylalkyl, C5-C12 heteroarylalkyl, or C1-C6 aminoalkyl group, each of which may be optionally substituted; or R₁ may cyclize with R_{1′} to form a 5-10 membered azacylic ring; and
each of R₂, R₃, R₄, and R₅ independently represents H, or C1-C8 alkyl, C2-C8 alkenyl, C2-C8 alkynyl, C1-C8 cycloalkylalkyl, C1-C8 heterocyclylalkyl, C1-C6 aminoalkyl, C5-C12 arylalkyl, or a heteroform of one of these, each of which may be optionally substituted;
with the proviso that the compound of formula (I) is not cyclo[-Phe-Leu-Val-Leu-Leu-] (SEQ ID NO: 1), or cyclo[-pBrPhe-Leu-Val-Leu-Leu-] (SEQ ID NO: 13), or a mono-N-methyl derivative thereof.

2. The compound of claim 1, wherein R₁ comprises CH₂ArX, where Ar represents a phenyl ring and X is selected from H, halo, OH, and C1-C4 alkoxy.

3. The compound of claim 1, wherein each of R₂, R₃, R₄ and R₅ independently represents H, methyl, ethyl, n-propyl, isopropyl, n-butyl, sec-butyl, isobutyl, tert-butyl, CH₂-cyclohexyl, CH₂OH, CH₂OBzl, or CH₂ArX, where Ar represents a phenyl ring and X is selected from H, halo, OH, and C1-C4 alkoxy.

4. The compound of claim 1, wherein each of R_{1′}, R_{2′}, R_{3′}, R_{4′}, and R_{5′} independently represents H or methyl.

5. The compound of claim 1, wherein the carbon atom bearing R₁ has the (R)-configuration, and the carbon atoms bearing R₂, R₃, R₄ and R₅ have the (S)-configuration.

6. The compound of claim 1, wherein the carbon atom bearing R₂ has the (R)-configuration, and the carbon atoms bearing R₁, R₃, R₄ and R₅ have the (S)-configuration.

7. The compound of claim 1, wherein the carbon atom bearing R₃ has the (R)-configuration, and the carbon atoms bearing R₁, R₂, R₄ and R₅ have the (S)-configuration.

8. The compound of claim 1, wherein the carbon atom bearing R₅ has the (R)-configuration, and the carbon atoms bearing R₁, R₂, R₃ and R₄ have the (S)-configuration.

9. The compound of claim 5, wherein R₁ is CH₂ArX, where Ar represents a phenyl ring and X is H, OH, OMe, Br or Cl.

10. The compound of claim 6, wherein R₂ is benzyl or isobutyl, and R_{2′} is H or Me.

11. The compound of claim 7, wherein R₃ is isopropyl or isobutyl, and R_{3′} is H or Me.

12. The compound of claim 8, wherein R₅ is isopropyl or isobutyl, and R₅ is H or Me.

13. The compound of claim 1, wherein any one of R_{1′}, R_{2′}, R_{3′}, R_{4′}, and R_{5′} is methyl, and the other four of R_{1′}, R_{2′}, R_{3′}, R_{4′}, and R_{5′} are H.

14. The compound of claim 13, wherein R_{2′} is methyl.

15. The compound of claim 13, wherein R_{3′} is methyl.

16. The compound of claim 13, wherein R_{5′} is methyl.

17. The compound of any of claims 1 through 12, wherein each of R_{1′}, R_{2′}, R_{3′}, R_{4′}, and R_{5′} is H.

18. The compound of claim 1, wherein two or more of the carbon atoms bearing R_{1′}, R_{2′}, R_{3′}, R_{4′}, and R_{5′} have the (R)-configuration.

19. The compound of claim 16, wherein the each of the carbon atoms bearing R_{1′}, R_{2′}, R_{3′}, R_{4′}, and R_{5′} have the (R)-configuration.

20. A compound of formula (II): 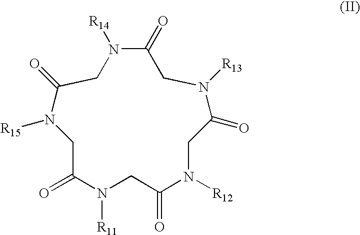
or a pharmaceutically acceptable salt or hydrate form thereof;
wherein each of R₁₁, R₁₂, R₁₃, R₁₄ and R₁₅ is independently selected from H, or C1-C8 alkyl, C2-C8 alkenyl, C2-C8 alkynyl, C1-C8 cycloalkylalkyl, C1-C6 aminoalkyl, C5-C12 arylalkyl, or a heteroform of one of these, each of which may be optionally substituted.

21. A pharmaceutical composition comprising one of more compounds of formula (I), or a pharmaceutically acceptable salt or hydrate form thereof, and a pharmaceutically acceptable excipient.

22. A pharmaceutical composition comprising one of more compounds of formula (II), or a pharmaceutically acceptable salt or hydrate form thereof, and a pharmaceutically acceptable excipient.

23. A method of treating, ameliorating or preventing (prophylaxis of) a condition comprising hyperplasia, unwanted cell growth or proliferation, or a cancer, comprising administering a therapeutically effective amount of a compound of claim 1, and/or the composition of claim 21 or claim 22, to a subject in need thereof,
and optionally the condition comprising hyperplasia, unwanted cell growth or proliferation, or a cancer, comprises a skin condition, psoriasis, a hormone-dependent tumor or a hormone-influenced non-malignant disorder, benign prostate hyperplasia (BPH), endometriosis; a disease or condition having an inflammatory component, an autoimmune disease, rheumatoid arthritis, an infectious disease, diabetic retinopathy, neovascular glaucoma, rheumatoid arthritis, psoriasis, a lung cancer, a lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma and/or any combination thereof.

24. The method of claim 23, wherein the cancer a colon cancer, MSS colon cancer, MSI colon cancer, pancreatic cancer, rectal cancer, breast cancer, prostate cancer, or melanoma.

25. The method of claim 23 or claim 24, wherein the subject is human or an animal.

26. A method to prepare a compound of formula (I), comprising macrocyclization of a linear pentapeptide of the formula (III) 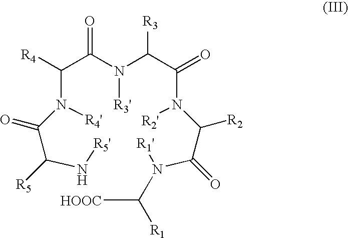
to provide a cyclic pentapeptide of formula (I)

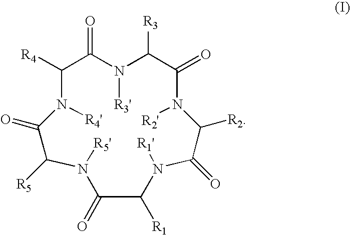

27. A method to prepare a compound of formula (II), comprising macrocyclization of a linear pentapeptoid of formula (IV) 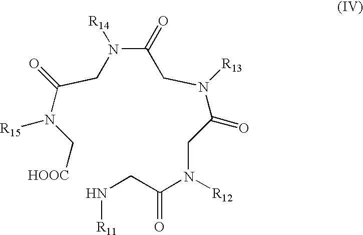
to provide a cyclic pentapeptoid of formula (II)

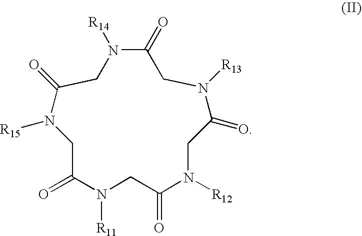

28. The method of claim 26 or 27, wherein said macrocyclization is performed by reaction with at least one coupling agent selected from the group HATU, DEPBT and TBTU.

29. The compound of claim 1 which is selected from:
(SEQ ID NO: 1)
cyclo[-Phe-Leu-Val-Leu-Leu-];
(SEQ ID NO: 18)
cyclo[-DPhe-DLeu-DVal-DLeu-DLeu-];
(SEQ ID NO: 2)
cyclo[-Phe-NMeLeu-Val-Leu-Leu-];
(SEQ ID NO: 18)
cyclo[-DPhe-DNMeLeu-DVal-DLeu-DLeu-];
(SEQ ID NO: 3)
cyclo[-Phe-NMeLeu-Val-Leu-NMeLeu-];
(SEQ ID NO: 18)
cyclo[-DPhe-DNMeLeu-DVal-DLeu-DNMeLeu-];
(SEQ ID NO: 4)
cyclo[-Phe-Leu-NMeVal-Leu-Leu-];
(SEQ ID NO: 18)
cyclo[-DPhe-DLeu-DNMeVal-DLeu-DLeu-];
SEQ ID NO: 5
cyclo[-Phe-Leu-Val-Leu-NMeLeu-]
SEQ ID NO: 6
cyclo[-Phe-Leu-NMeVal-Leu-NMeLeu-]
(SEQ ID NO: 18)
cyclo[-DPhe-Leu-DNMeVal-DLeu-DNMeLeu-];
(SEQ ID NO: 7)
cyclo[-Phe-NMeLeu-NMeVal-Leu-NMeLeu-];
(SEQ ID NO: 18)
cyclo[-DPhe-DNMeLeu-DNMeVal-DLeu-DNMeLeu-];
(SEQ ID NO: 8)
cyclo[-Phe-NMeLeu-NMeVal-Leu-Leu-];
(SEQ ID NO: 9)
cyclo[-Phe-Gly-Val-Leu-Leu-];
(SEQ ID NO: 2)
cyclo[-Phe-Sar-Val-Leu-Leu-];
(SEQ ID NO: 10)
cyclo[-Phe-Leu-Ala-Leu-Leu-];
(SEQ ID NO: 4)
cyclo[-Phe-Leu-(αEt)Gly-Leu-Leu-];
(SEQ ID NO: 11)
cyclo[-Phe-Ile-Val-Leu-Leu-];
(SEQ ID NO: 12)
cyclo[-Phe-Ala-Val-Leu-Leu-];
(SEQ ID NO: 13)
cyclo[-DPhe-Leu-Val-Leu-Leu-];
(SEQ ID NO: 2)
cyclo[-Phe-DLeu-Val-Leu-Leu-];
(SEQ ID NO: 4)
cyclo[-Phe-Leu-DVal-Leu-Leu-];
(SEQ ID NO: 17)
cyclo[-Phe-Leu-Val-DLeu-Leu-];
(SEQ ID NO: 25)
cyclo[-DPhe-DLeu-Val-Leu-Leu-];
(SEQ ID NO: 26)
cyclo[-DPhe-DLeu-DVal-Leu-Leu-];
(SEQ ID NO: 27)
cyclo[-DPhe-DLeu-DVal-DLeu-Leu-];
(SEQ ID NO: 13)
cyclo[-NMePhe-Leu-Val-Leu-Leu-];
(SEQ ID NO: 14)
cyclo[-NMePhe-Leu-NMeVal-Leu-Leu-];
(SEQ ID NO: 4)
cyclo[-Phe-Leu-DNMeVal-Leu-Leu-];
(SEQ ID NO: 2)
cyclo[-Phe-DNMeLeu-Val-Leu-Leu-];
(SEQ ID NO: 5)
cyclo[-Phe-Leu-Val-Leu-DLeu-];
(SEQ ID NO: 17)
cyclo[-Phe-Leu-Val-NMeLeu-Leu-];
(SEQ ID NO: 15)
cyclo[-Phe-Leu-Val-Ile-Leu-];
(SEQ ID NO: 16)
cyclo[-Phe-Val-Leu-Leu-Val-];
(SEQ ID NO: 2)
cyclo[-Phe-nBug-Val-Leu-Leu-];
(SEQ ID NO: 19)
cyclo[-Phe-Leu-Val-NMeLeu-DLeu-];
(SEQ ID NO: 30)
cyclo[-DPhe-Leu-Val-NMeLeu-DLeu-];
(SEQ ID NO: 13)
cyclo[-Tic-Leu-Val-Leu-Leu-];
(SEQ ID NO: 4)
cyclo[-Phe-Leu-DLeu-Leu-Leu-];
(SEQ ID NO: 28)
cyclo[-DPhe-Val-DLeu-DLeu-Ile-];
(SEQ ID NO: 19)
cyclo[-Phe-Leu-Val-DLeu-DLeu-];
(SEQ ID NO: 29)
cyclo[-Phe-Leu-DVal-DLeu-DLeu-];
(SEQ ID NO: 8)
cyclo[-Phe-DNMeLeu-DNMeVal-Leu-Leu-];
(SEQ ID NO: 20)
cyclo[-Phe-Val-DVal-Leu-Sar-];
(SEQ ID NO: 21)
cyclo[-Phe-Leu-DNMeVal-Cha-Leu-];
(SEQ ID NO: 13)
cyclo[-DTyr-Leu-Val-Leu-Leu-];
(SEQ ID NO: 13)
cyclo[-DTrp-Leu-Val-Leu-Leu-];
(SEQ ID NO: 4)
cyclo[-Phe-Leu-DSer-Leu-Leu-];
(SEQ ID NO: 5)
cyclo[-Phe-Leu-Val-Leu-DNMeLeu-];
(SEQ ID NO: 2)
cyclo[-Phe-DNMePhe-Val-Leu-Leu-];
(SEQ ID NO: 2)
cyclo[-Phe-DPhe-Val-Leu-Leu-];
(SEQ ID NO: 22)
cyclo[-Phe-Val-DVal-Val-Val-];
(SEQ ID NO: 22)
cyclo[-Phe-Val-D(αEt)Gly-Val-Val-];
(SEQ ID NO: 22)
cyclo[-Phe-Val-DSer-Val-Val-];
(SEQ ID NO: 23)
cyclo[-Phe-DSer-Val-Val-Val-];
(SEQ ID NO: 4)
cyclo[-Phe-Leu-DPhe-Leu-Leu-];
(SEQ ID NO: 2)
cyclo[-Phe-DSer-Val-Leu-Leu-];
(SEQ ID NO: 24)
cyclo[-Phe-Val-DNMeVal-DSer-Leu-];
(SEQ ID NO: 8)
cyclo[-Phe-DLeu-(αEt)Gly-Leu-Leu-];
(SEQ ID NO: 4)
cyclo[-Phe-Leu-D(αEt)Gly-Leu-Leu-];
(SEQ ID NO: 8)
cyclo[-Phe-DLeu-DVal-Leu-Leu-];
(SEQ ID NO: 6)
cyclo[-Phe-Leu-DNMeVal-Leu-DLeu-];
(SEQ ID NO: 31)
cyclo[-Tyr-Leu-DNMeVal-Leu-Leu-];
(SEQ ID NO: 32)
cyclo[-Val-Leu-DNMeVal-Leu-Leu-];
(SEQ ID NO: 6)
cyclo[-Phe-Leu-DNMeVal-Leu-NMeLeu-];
(SEQ ID NO: 6)
cyclo[-Phe-Leu-DVal-Leu-DLeu-];
(SEQ ID NO: 6)
cyclo[-Phe-Leu-DVal-Leu-NMeLeu-];
(SEQ ID NO: 13)
cyclo[-DpBrPhe-Leu-Val-Leu-Leu-];
(SEQ ID NO: 14)
cyclo[-DPhe-Leu-DNMeVal-Leu-Leu-];
(SEQ ID NO: 26)
cyclo[-DPhe-DLeu-DNMeVal-Leu-Leu-];
(SEQ ID NO: 14)
cyclo[-DPhe-Leu-DVal-Leu-Leu-];
(SEQ ID NO: 14)
cyclo[-DPhe-Leu-DLys-Leu-Leu-];
(SEQ ID NO: 33)
cyclo[-Phe-Leu-DVal-Lys-Leu-];
(SEQ ID NO: 34)
cyclo[-DPhe-DLeu-DVal-Lys-Leu-];
(SEQ ID NO: 35)
cyclo[-Phe-Leu-Val-Lys-Leu-];
(SEQ ID NO: 21)
cyclo[-Phe-Leu-D(αEt)Gly-Cha-Leu-];
(SEQ ID NO: 36)
cyclo[-Phe-Leu-DNMeVal-Leu-Lys-];
(SEQ ID NO: 4)
cyclo[-Phe-Leu-DLys-Leu-Leu-];
(SEQ ID NO: 37)
cyclo[-DTyr-Leu-Val-Lys-Leu-];
(SEQ ID NO: 38)
cyclo[-DTrp-Leu-Val-Arg-Leu-];
(SEQ ID NO: 39)
cyclo[-Lys-Leu-Val-Leu-Leu-];
(SEQ ID NO: 26)
cyclo[-DPhe-DSer-DLys-Leu-Leu-];
(SEQ ID NO: 40)
cyclo[-Lys-Leu-DVal-Leu-Leu-];
(SEQ ID NO: 26)
cyclo[-DLys-DLeu-DVal-Leu-Leu-];
(SEQ ID NO: 22)
cyclo[-Phe-Val-D(OBzl)Ser-Val-Val-];
(SEQ ID NO: 23)
cyclo[-Phe-D(OBzl)Ser-Val-Val-Val-];
(SEQ ID NO: 21)
cyclo[-Phe-Leu-DVal-CBZLys-Leu-];
(SEQ ID NO: 27)
cyclo[-DPhe-DLeu-DVal-CBzLys-Leu-];
(SEQ ID NO: 17)
cyclo[-Phe-Leu-Val-CBZLys-Leu-];
(SEQ ID NO: 41)
cyclo[-DPhe-DLeu-DVal-Leu-Ser-];
(SEQ ID NO: 42)
cyclo[-DPhe-DLeu-DVal-Leu-Lys-];
(SEQ ID NO: 43)
cyclo[-Phe-DNMePhe-Val-Leu-Ser-];
(SEQ ID NO: 44)
cyclo[-Phe-DNMePhe-Val-Leu-Lys-];
(SEQ ID NO: 45)
cyclo[-Phe-DNMePhe-Val-Cha-Ser-];
(SEQ ID NO: 46)
cyclo[-Phe-DNMePhe-DVal-Cha-Ser-];
(SEQ ID NO: 47)
cyclo[-Phe-DNMePhe-Val-CBZLys-Leu-];
(SEQ ID NO: 48)
cyclo[-Phe-DNMePhe-Val-Lys-Leu-];
(SEQ ID NO: 25)
cyclo[-DTyr-DNMePhe-Val-Leu-Leu-];
(SEQ ID NO: 21)
cyclo[-Phe-Leu-DNMeVal-DLeu-Leu-];
(SEQ ID NO: 6)
cyclo[-Phe-Leu-DNMeVal-Leu-CBZLys-];
(SEQ ID NO: 49)
cyclo[-Phe-DNMePhe-Val-Cha-(OBzl)Ser-].
(SEQ ID NO: 50)
cyclo[-Phe-DNMePhe-DVal-Cha-(OBzl)Ser-];
(SEQ ID NO: 51)
cyclo[-DNMePhe-Leu-Val-Leu-DTyr-];
(SEQ ID NO: 13)
cyclo[-DNMeTyr-Leu-Val-Leu-Leu-];
(SEQ ID NO: 14)
cyclo[-DNMeTyr-Leu-DVal-Leu-Leu-];
(SEQ ID NO: 52)
cyclo[-DTyr-DNMePhe-Val-NMeLeu-Leu-];
and
(SEQ ID NO: 53)
cyclo[-DTyr-DNMePhe-Val-NMeLeu-DLeu-].

30-31. (canceled)

32. A pharmaceutical composition or dietary supplement formulated as a tablet, gel, geltab, pill, implant, liquid, spray, powder, food, feed pellet, as an injectable formulation or as an encapsulated formulation, lotion, patch or inhalant, and at least one compound of claim 1 or claim 29, and/or a compound made by the method of any of claims 26 to 28.
